SEQUENCE LISTING (98 sequences)

SEQ ID NO: 1 - DNA, 31 bp, Artificial Sequence
  TP53 reference sequence
agccaagtctgtaacctgcacgtactcccct

SEQ ID NO: 2 - DNA, 14 bp, Artificial Sequence
  TP53 KO#1
agccgtactcccct

SEQ ID NO: 3 - DNA, 27 bp, Artificial Sequence
  TP53 KO#2
agccaagtctgtaacctgcactcccct

SEQ ID NO: 4 - DNA, 29 bp, Artificial Sequence
  KRAS G12D reference sequence
cgatagaggattcctacaggaaacaagta

SEQ ID NO: 5 - DNA, 28 bp, Artificial Sequence
  KRAS G12D INDEL
cgatagaggattctacaggaaacaagta

SEQ ID NO: 6 - DNA, 29 bp, Artificial Sequence
  KRAS G12D INDEL
cgatagaggattccctacaggaaacaagt

SEQ ID NO: 7 - DNA, 29 bp, Artificial Sequence
  KRAS G12D INDEL
cgatagaggattcctacaggaaacaagta

SEQ ID NO: 8 - DNA, 18 bp, Artificial Sequence
  KRAS G12D INDEL
cgatagaggaaacaagta

SEQ ID NO: 9 - DNA, 25 bp, Artificial Sequence
  KRAS G12D INDEL
cgatagaggatacaggaaacaagta

SEQ ID NO: 10 - DNA, 18 bp, Artificial Sequence
  KRAS G12D INDEL
cgatacaggaaacaagta

SEQ ID NO: 11 - DNA, 26 bp, Artificial Sequence
  KRAS G12D INDEL
cgatagaggattacaggaaacaagta

SEQ ID NO: 12 - DNA, 25 bp, Artificial Sequence
  KRAS G12D INDEL
cgatagaggattcaggaaacaagta

SEQ ID NO: 13 - DNA, 28 bp, Artificial Sequence
  KRAS G12D INDEL
cgatagaggattccacaggaaacaagta

SEQ ID NO: 14 - DNA, 18 bp, Artificial Sequence
  KRAS G12D INDEL
cgatagaggattcaagta

SEQ ID NO: 15 - DNA, 27 bp, Artificial Sequence
  KRAS G12D INDEL
cgatagaggatttacaggaaacaagta

SEQ ID NO: 16 - DNA, 27 bp, Artificial Sequence
  KRAS G12D INDEL
cgatagaggatctacaggaaacaagta

SEQ ID NO: 17 - DNA, 29 bp, Artificial Sequence
  KRAS G12D INDEL
cgatagaggattccttacaggaaacaagt

SEQ ID NO: 18 - DNA, 24 bp, Artificial Sequence
  KRAS G12D INDEL
cgatagaggtacaggaaacaagta

SEQ ID NO: 19 - DNA, 26 bp, Artificial Sequence
  KRAS G12D INDEL
cgatagaggattccaggaaacaagta

SEQ ID NO: 20 - DNA, 29 bp, Artificial Sequence
  TP53 R167H reference
gtctgtaacctgcacgtactcccctgccc

SEQ ID NO: 21 - DNA, 29 bp, Artificial Sequence
  TP53 R167H INDEL
gtctgtaacctgcacgtactcccctgccc

SEQ ID NO: 22 - DNA, 15 bp, Artificial Sequence
  TP53 R167H INDEL
gtctgtaacctgccc

SEQ ID NO: 23 - DNA, 27 bp, Artificial Sequence
  TP53 R167H INDEL
gtctgtaacctgcatactcccctgccc

SEQ ID NO: 24 - DNA, 29 bp, Artificial Sequence
  TP53 R167H INDEL
gtctgtaacctgcaacgtactcccctgcc

SEQ ID NO: 25 - DNA, 29 bp, Artificial Sequence
  TP53 R167H INDEL
gtctgtaacctgcaccgtactcccctgcc

SEQ ID NO: 26 - DNA, 27 bp, Artificial Sequence
  TP53 R167H INDEL
gtctgtaacctgcgtactcccctgccc

SEQ ID NO: 27 - DNA, 28 bp, Artificial Sequence
  TP53 R167H INDEL
gtctgtaacctgcagtactcccctgccc

SEQ ID NO: 28 - DNA, 29 bp, Artificial Sequence
  TP53 R167H INDEL
gtctgtaacctgcatcgtactcccctgcc

SEQ ID NO: 29 - DNA, 25 bp, Artificial Sequence
  TP53 R167H INDEL
gtctgtaacctgcactcccctgccc

SEQ ID NO: 30 - DNA, 18 bp, Artificial Sequence
  TP53 R167H INDEL
gtctgtactcccctgccc

SEQ ID NO: 31 - DNA, 25 bp, Artificial Sequence
  TP53 R167H INDEL
gtctgtaacctgtactcccctgccc

SEQ ID NO: 32 - DNA, 23 bp, Artificial Sequence
  TP53 R167H INDEL
gtctgtaacgtactcccctgccc

SEQ ID NO: 33 - DNA, 23 bp, Artificial Sequence
  TP53 R167H INDEL
gtctgtaacctgcacccctgccc

SEQ ID NO: 34 - DNA, 27 bp, Artificial Sequence
  TP53 R167H INDEL
gtctgtaacctgcagactcccctgccc

SEQ ID NO: 35 - DNA, 24 bp, Artificial Sequence
  TP53 R167H INDEL
gtctgtaacctgcatcccctgccc

SEQ ID NO: 36 - DNA, 24 bp, Artificial Sequence
  KRAS/TP53 deletion region
atagaggattcccgtactcccctg

SEQ ID NO: 37 - DNA, 40 bp, Artificial Sequence
  ARID1A sequence
agcagggccagactccatattacaaccagcaaagtcctca

SEQ ID NO: 38 - DNA, 40 bp, Artificial Sequence
  ARID1A INDEL
agcagggccagactccatatttacaaccagcaaagtcctc

SEQ ID NO: 39 - DNA, 39 bp, Artificial Sequence
  ARID1A INDEL
agcagggccagactccatatacaaccagcaaagtcctca

SEQ ID NO: 40 - DNA, 40 bp, Artificial Sequence; identical to SEQ ID NO: 37
  ARID1A INDEL

SEQ ID NO: 41 - DNA, 37 bp, Artificial Sequence
  ARID1A INDEL
agcagggccagactccatacaaccagcaaagtcctca

SEQ ID NO: 42 - DNA, 38 bp, Artificial Sequence
  ARID1A INDEL
agcagggccagactccattacaaccagcaaagtcctca

SEQ ID NO: 43 - DNA, 29 bp, Artificial Sequence
  ARID1A INDEL
agcagggccagactccagcaaagtcctca

SEQ ID NO: 44 - DNA, 40 bp, Artificial Sequence
  ARID1A INDEL
agcagggccagactccatatatacaaccagcaaagtcctc

SEQ ID NO: 45 - DNA, 22 bp, Artificial Sequence
  ARID1A INDEL
agcagggccagcaaagtcctca

SEQ ID NO: 46 - DNA, 38 bp, Artificial Sequence
  ARID1A INDEL
agcagggccagactccatatcaaccagcaaagtcctca

SEQ ID NO: 47 - DNA, 30 bp, Artificial Sequence
  ARID1A INDEL
agcagggccagacaaccagcaaagtcctca

SEQ ID NO: 48 - DNA, 33 bp, Artificial Sequence
  ARID1A INDEL
agcagggccagactccaaccagcaaagtcctca

SEQ ID NO: 49 - DNA, 36 bp, Artificial Sequence
  ARID1A INDEL
agcagggccagactccatataccagcaaagtcctca

SEQ ID NO: 50 - DNA, 13 bp, Artificial Sequence
  ARID1A INDEL
agcaaagtcctca

SEQ ID NO: 51 - DNA, 40 bp, Artificial Sequence
  ARID1A INDEL
agcagggccagactccatatgtacaaccagcaaagtcctc

SEQ ID NO: 52 - DNA, 27 bp, Artificial Sequence
  ARID1A INDEL
agcagggccagaccagcaaagtcctca

SEQ ID NO: 53 - DNA, 37 bp, Artificial Sequence
  ARID1A INDEL
agcagggccagactccatataaccagcaaagtcctca

SEQ ID NO: 54 - DNA, 36 bp, Artificial Sequence
  ARID1A INDEL
agcagggccagactcctacaaccagcaaagtcctca

SEQ ID NO: 55 - DNA, 10 bp, Artificial Sequence
  ARID1A INDEL
aaagtcctca

SEQ ID NO: 56 - DNA, 34 bp, Artificial Sequence
  ARID1A INDEL
agcagggccagactccataccagcaaagtcctca

SEQ ID NO: 57 - DNA, 35 bp, Artificial Sequence
  ARID1A INDEL
agcagggccagactccataaccagcaaagtcctca

SEQ ID NO: 58 - DNA, 37 bp, Artificial Sequence
  ARID1A INDEL
agcagggccagactccatattaccagcaaagtcctca

SEQ ID NO: 59 - DNA, 35 bp, Artificial Sequence
  ARID1A INDEL
agcagggccagactccacaaccagcaaagtcctca

SEQ ID NO: 60 - DNA, 40 bp, Artificial Sequence
  ARID1A INDEL
agcagggccagactccatatctacaaccagcaaagtcctc

SEQ ID NO: 61 - DNA, 38 bp, Artificial Sequence
  ARID1A INDEL
agcagggccagactccatattaaccagcaaagtcctca

SEQ ID NO: 62 - DNA, 33 bp, Artificial Sequence
  ARID1A INDEL
agcagggccagactacaaccagcaaagtcctca

SEQ ID NO: 63 - DNA, 40 bp, Artificial Sequence
  ARID1A INDEL
agcagggccagactccatattatacaaccagcaaagtcct

SEQ ID NO: 64 - DNA, 37 bp, Artificial Sequence
  ARID1A INDEL
agcagggccagactccagacaaccagcaaagtcctca

SEQ ID NO: 65 - DNA, 30 bp, Artificial Sequence
  ARID1A INDEL
agcagggccagactccatataaagtcctca

SEQ ID NO: 66 - DNA, 16 bp, Artificial Sequence
  ARID1A INDEL
agcagcaaagtcctca

SEQ ID NO: 67 - DNA, 40 bp, Artificial Sequence
  ARID1A INDEL
agcagggccagactccatatacatacaaccagcaaagtcc

SEQ ID NO: 68 - DNA, 18 bp, Artificial Sequence
  ARID1A INDEL
agcagggccagactccat

SEQ ID NO: 69 - DNA, 40 bp, Artificial Sequence
  AXIN1 refernce sequence
cgagtcgctgcactccctgctcgacgatcaggacgggata

SEQ ID NO: 70 - DNA, 40 bp, Artificial Sequence
  AXIN1 INDEL
cgagtcgctgcactccctgcttcgacgatcaggacgggat

SEQ ID NO: 71 - DNA, 40 bp, Artificial Sequence; identical to SEQ ID NO: 69
  AXIN1 INDEL

SEQ ID NO: 72 - DNA, 38 bp, Artificial Sequence
  AXIN1 INDEL
cgagtcgctgcactccctgcgacgatcaggacgggata

SEQ ID NO: 73 - DNA, 33 bp, Artificial Sequence
  AXIN1 INDEL
cgagtcgctgcactcgacgatcaggacgggata

SEQ ID NO: 74 - DNA, 24 bp, Artificial Sequence
  AXIN1 INDEL
cgagtcgacgatcaggacgggata

SEQ ID NO: 75 - DNA, 35 bp, Artificial Sequence
  AXIN1 INDEL
cgagtcgctgcactcccgacgatcaggacgggata

SEQ ID NO: 76 - DNA, 40 bp, Artificial Sequence
  AXIN1 INDEL
cgagtcgctgcactccctgcatcgacgatcaggacgggat

SEQ ID NO: 77 - DNA, 38 bp, Artificial Sequence
  AXIN1 INDEL
cgagtcgctgcactccctgcagcgatcaggacgggata

SEQ ID NO: 78 - DNA, 40 bp, Artificial Sequence
  AXIN1 INDEL
cgagtcgctgcactccctgcagcgacgatcaggacgggat

SEQ ID NO: 79 - DNA, 35 bp, Artificial Sequence
  AXIN1 INDEL
cgagtcgctgcactccctgcgatcaggacgggata

SEQ ID NO: 80 - DNA, 39 bp, Artificial Sequence
  AXIN1 INDEL
cgagtcgctgcactccctgtcgacgatcaggacgggata

SEQ ID NO: 81 - DNA, 39 bp, Artificial Sequence
  AXIN1 INDEL
cgagtcgctgcactccctgccgacgatcaggacgggata

SEQ ID NO: 82 - DNA, 40 bp, Artificial Sequence
  AXIN1 INDEL
cgagtcgctgcactccctgcctcgacgatcaggacgggat

SEQ ID NO: 83 - DNA, 40 bp, Artificial Sequence
  AXIN1 INDEL
cgagtcgctgcactccctgatcgacgatcaggacgggata

SEQ ID NO: 84 - DNA, 38 bp, Artificial Sequence
  AXIN1 INDEL
cgagtcgctgcactcccttcgacgatcaggacgggata

SEQ ID NO: 85 - DNA, 39 bp, Artificial Sequence
  AXIN1 INDEL
cgagtcgctgcactccctgctgacgatcaggacgggata

SEQ ID NO: 86 - DNA, 19 bp, Artificial Sequence
  AXIN1 INDEL
cgagtcgctgcactccctg

SEQ ID NO: 87 - DNA, 36 bp, Artificial Sequence
  AXIN1 INDEL
cgagtcgctgcactccctgacgatcaggacgggata

SEQ ID NO: 88 - DNA, 28 bp, Artificial Sequence
  AXIN1 INDEL
cgagtcgctgcacgatcaggacgggata

SEQ ID NO: 89 - DNA, 40 bp, Artificial Sequence
  AXIN1 INDEL
cgagtcgctgcactccctgcactcgacgatcaggacggga

SEQ ID NO: 90 - DNA, 32 bp, Artificial Sequence
  AXIN1 INDEL
cgagtcgctgcactcccgatcaggacgggata

SEQ ID NO: 91 - DNA, 20 bp, Artificial Sequence
  AXIN1 INDEL
cgagtcgctgcactccctgc

SEQ ID NO: 92 - DNA, 37 bp, Artificial Sequence
  AXIN1 INDEL
cgagtcgctgcactccctgcacgatcaggacgggata

SEQ ID NO: 93 - DNA, 21 bp, Artificial Sequence
  AXIN1 INDEL
cgagtcgatcaggacgggata

SEQ ID NO: 94 - DNA, 18 bp, Artificial Sequence
  ARID1A reference sequence
gactccatattacaacca

SEQ ID NO: 95 - DNA, 8 bp, Artificial Sequence
  ARID1A knockout
gacaacca

SEQ ID NO: 96 - DNA, 17 bp, Artificial Sequence
  ARID1A knockout
gactccatatacaacca

SEQ ID NO: 97 - DNA, 17 bp, Artificial Sequence
  ARID1A knockout
gactccatatacaacca

SEQ ID NO: 98 - DNA, 18 bp, Artificial Sequence
  ARID1A knockout
gactccatatttacaacc

(19) United States
(12) Patent Application Publication
(10) Pub. No.: US 2022/0186190 A1
(43) Pub. Date: Jun. 16, 2022

(54) MODELING ONCOLOGY ON DEMAND

(71) Applicant: The Board of Trustees of the University of Illinois, Urbana, IL (US)

(72) Inventors: Lawrence B. Schook, Chicago, IL (US); Ron C. Gaba, Chicago, IL (US); Regina M. Schwind, Chicago, IL (US); Kyle Schachtschneider, Chicago, IL (US)

(73) Assignee: The Board of Trustees of the University of Illinois, Urbana, IL (US)

(21) Appl. No.: 17/436,441

(22) PCT Filed: Mar. 3, 2020
(86) PCT No.: PCT/US2020/020837

§ 371 Date: Sep. 3, 2021

Related U.S. Application Data
(60) Provisional application No. 62/813,307, filed on Mar. 4, 2019.

(51) Int. Cl. C12N 5/09; A01K 67/027; A23K 20/105

(52) U.S. Cl. CPC C12N 5/0693; A01K 67/0275; A23K 20/105; A01K 2227/108; A01K 2217/05; A01K 2207/00; A01K 2267/0331; A01K 2267/0381; A01K 2217/203

(57) ABSTRACT

Genetically modified pigs having at least one cancer and/or at least one co-morbid condition are provided. Also provided are methods of using the pig and derived tumor cells to screen for therapeutic compounds, medical devices or procedures, and/or combinations thereof. Further provided are methods of producing personalized cancer models, including obtaining a tumor sample from a subject, identifying mutations in the tumor sample, and producing a genetically modified tumor or tumor cell line having the same mutations.

### CROSS REFERENCE TO RELATED APPLICATION

This application claims the benefit of U.S. Provisional Application No. 62/813,307, filed Mar. 4, 2019, which is incorporated by reference herein in its entirety.

### ACKNOWLEDGMENT OF GOVERNMENT SUPPORT

This invention was made with government support under grant number 1-R21-CA-195433-01A1 awarded by the National Institutes of Health and Translational Team Science Award W81XWH-16-1-0335 awarded by the Department of Defense. The government has certain rights in the invention.

### FIELD

This disclosure relates to pig models of cancer and methods of their use, particularly for testing therapeutics.

### BACKGROUND

The global incidence of cancer is rapidly rising, and despite an improved understanding of cancer molecular biology, immune landscapes, and advancements in cytotoxic, biologic, and immunologic anti-cancer therapeutics, cancer remains a leading cause of death worldwide. While human clinical trials are the benchmark for advancing clinical standard-of-care therapeutics, the regulatory, financial, and enrollment burdens of trial conduct are substantial. First, only 1 in 1,000 potential drugs graduate to human clinical trials, and of these, 90% fail in human testing. Furthermore, the time and cost associated with taking a new drug from concept to market approximates 12 years and exceeds $1.3 billion, respectively; medical devices are associated with similarly protracted, 3-7 year approval periods costing tens of millions of dollars. Finally, the pool of available patients is not large enough to support the thousands of ongoing clinical trials, resulting in competition between trials, slow accrual, or early trial closure. This is particularly true for precision medicine trials; new trial designs (such as adaptive, basket, umbrella designs) for assessment of precision medicine interventions are limited by a complexity that lengthens the activation process while increasing the number of screen-failed participants, all of which hinder the generation of targeted gene and molecular therapies for cancer.

Beyond cancer alone, patients also suffer from a rising number of medical comorbidities that increase and/or potentiate cancer risk, affect cancer growth and development, influence cancer stage at diagnosis, impact cancer treatment, and affect cancer clinical outcomes. For example, patients with diabetes are at increased risk for several cancer types, and liver cirrhosis restricts therapeutic options for liver cancer given the potential for collateral damage to the diseased liver, which serves as a competing cause of death. At present, such comorbidities are poorly captured in clinical trials, where stringent eligibility criteria hinder accrual and jeopardize the generalizability of study results to patients at large exhibiting relevant medical comorbidities and conditions. Development of new standards of care is thus often impeded by the exclusion of patients with comorbidities from gold standard trials.

Currently, standard small and large animal models are used for preclinical testing of anticancer drugs and devices, however each existing animal model system has significant shortcomings which preclude faithful recapitulation of human malignancies in their appropriate host environment(s). Small laboratory animals (such as mice, rats, woodchucks, and rabbits), are limited by anatomic, physiologic, metabolic, and genetic differences compared to humans, which preclude translation of preclinical findings to the clinic. Another major drawback of small animal models is their diminutive size, which prevents clinical device utilization and limits technical capability for clinically translatable interventions. Recent literature has also uncovered meaningful issues with reproducibility of findings made in murine systems. Rabbit cancer models are limited by a narrow breadth of tumor cell types, unknown tumor biology and kinetics, and unknown genome organization. In addition, despite feasibility of producing tumors in rabbit models, the tumors have a tendency towards spontaneous necrosis.

Large animal models include dogs and non-human primates. Disadvantages of the canine cancer models include predominantly lymphoid and sarcoma tumor types as opposed to carcinomas, lack of translatability of cancer drug studies due to varying drug sensitivity in dogs compared to humans, inability of inbred species to recapitulate human cancer heterogeneity, and the challenge of accrual of client dogs to clinical trials. In regards to non-human primates, the concordance of toxicities identified in these animals relative to humans and other species is unclear because of a lack of data. Another major limitation of these large animal models is the looming exclusion of non-human primates and companion animals from translational research due to ethical considerations. All of the described animal models generally lack the capability to experimentally produce relevant medical comorbidities concurrent to tumor generation, limiting the ability to reflect real-life clinical scenarios.

Another strategy in preclinical testing consists of utilizing patient-derived xenografts (PDXs), generated by direct implantation of surgically resected tumor samples into mice. However, in addition to the above-mentioned limitations of small animal models, PDX tumors are often grown subcutaneously as opposed to their orthotopic site of origin, do not progress or metastasize, and display altered tumor characteristics and genetic integrity compared to the tumor of origin over time. Moreover, PDX models require mice with severely compromised immune systems, and can take up to a year to develop, limiting their usefulness in co-clinical trials and personalized medicine. Effective preclinical animal models for cancers and their associated comorbidities are thus critical in overcoming these challenges.

### SUMMARY

Disclosed herein are animal models and methods of use that can efficiently and effectively undergo clinical trial participation without enrollment, sex, pregnancy, age, or comorbidity hurdles, successfully eliminating logistical and accrual clinical trial barriers. Additionally, such animal models permit prospective early phase assessment of new modalities and facilitate validation of novel technologies, such as nanotherapy, gene therapy, and immunotherapy, as well as high efficiency and ethical conduct of cohort driven studies either preceding or in conjunction with human clinical trials that afford the ability to screen and funnel promising therapies prior to human testing.

In some embodiments, disclosed herein are in vitro methods for screening for therapeutic compounds. The methods include contacting cells from a pig having at least one cancer and at least one co-morbid condition with one or more test compounds, measuring cell growth and/or survival, and identifying a compound as a therapeutic compound if the cell growth and/or survival is decreased compared to a control. In particular examples, the cells are derived pig for Cre recombinase-inducible human driver mutations (e.g., KRAS^{G12D} and TP53^{R167H}). In other examples, the methods include collecting cells from a pig that has one or more co-morbid condition, but does not have a tumor. The cells are then transformed in vitro and contacted with one or more test compounds. Cell growth and/or survival is measured and a compound is identified as a therapeutic compound if the cell growth and/or survival is decreased compared to a control.

In other embodiments, disclosed herein are in vivo methods for screening for therapeutic compounds. The methods include administering one or more test compounds to a pig having at least one cancer and at least one co-morbid condition, determining the effect of the one or more test compounds on the at least one cancer and/or the at least one co-morbid condition, and identifying a compound as a therapeutic compound if the at least one cancer and/or the at least one co-morbid condition is ameliorated compared to a control. In some examples, the pig is transgenic for Cre recombinase-inducible KRAS^{G12D} and TP53^{R167H}. In some embodiments, the methods further include administering one or more medical devices or procedures to the pig that is administered the one or more test compounds.

In further embodiments, disclosed are methods of testing medical devices or procedures. The methods include administering one or more medical device or procedure to a pig having at least one cancer and at least one co-morbid condition, and determining the effect of the medical device or procedure on the at least one cancer and/or the at least one co-morbid condition compared to a control. In some examples, the pig is transgenic for Cre recombinase-inducible KRAS^{G12D} and TP53^{R167H}. In particular examples, the medical device or procedure includes one or more of imaging, thermal therapy, targeted drug activation, targeted drug delivery, and embolization (including embolization with or without one or more chemotherapeutic agents).

In the disclosed embodiments, the pig may have one or more cancers, including but not limited to liver cancer, sarcoma, lung cancer, colorectal cancer, esophageal cancer, pancreatic cancer, breast cancer, bladder cancer, kidney cancer, endometrial cancer, ovarian cancer, hematological cancer, and metastatic disease. In addition, the pig may have one or more co-morbid conditions, including but not limited to one or more of cirrhosis, diabetes, obesity, hyperlipidemia, metabolic syndrome, cardiovascular disease, non-alcoholic fatty liver disease, non-alcoholic steatohepatitis, chronic kidney disease, high-fat diet, tobacco use, alcohol use, caffeine use, and marijuana use. In some examples, the combination of at least one cancer and at least one co-morbid condition is not liver cancer (e.g., hepatocellular carcinoma) and any one of cirrhosis, obesity, and non-alcoholic steatohepatitis.

In other embodiments, disclosed are methods of producing a personalized cancer model. The methods include obtaining a tumor sample from a subject (such as a subject with cancer, e.g., a human subject with cancer), sequencing the genome of tumor cells from the tumor sample, identifying one or more mutations in the genomic sequence of the tumor cells (e.g., one or more driver mutations), introducing the one or more mutations into isolated cells from a pig, to produce isolated pig cells comprising the one or more mutations, and administering the isolated pig cells comprising the one or more mutations to a pig to produce one or more tumors in the pig. In other examples, the method includes introducing the one or more mutations identified in the tumor cells directly in the pig (e.g., utilizing in vivo gene editing) to produce one or more tumors in the pig. In some examples, the pig has one or more co-morbid conditions. In particular examples, the pig is transgenic for Cre recombinase-inducible KRAS^{G12D} and TP53^{R167H}. In some examples, the methods include administering one or more treatments to the pig having one or more tumors, such as administering one or more test compounds (e.g., one or more cytotoxic or chemotherapeutic agents) and/or administering one or more medical device or procedure to the pig having one or more tumors. If the one or more therapeutic compound and/or one or more medical device or procedure ameliorates the cancer and/or the co-morbid condition, the one or more therapeutic compound and/or one or more medical device or procedure is administered to the subject.

Also disclosed are methods of identifying one or more diagnostic or prognostic biomarkers, for example one or more biomarkers diagnostic or prognostic for at least one cancer and/or at least one co-morbid condition. In some examples, the methods include obtaining one or more samples from a pig having at least one cancer and at least one co-morbid condition, measuring levels of one or more biomarkers in the sample, and comparing the one or more biomarker with a control. In some examples, the pig is transgenic for Cre recombinase-inducible KRAS^{G12D} and TP53^{R167H}.

Disclosed herein are transgenic pig with at least one cancer and at least one co-morbid condition, wherein the pig is transgenic for Cre recombinase-inducible KRAS^{G12D} and TP53^{R167H}. In some non-limiting examples, the combination of at least one cancer and at least one co-morbid condition is not liver cancer (e.g., hepatocellular carcinoma) and any one of cirrhosis, obesity, and non-alcoholic steatohepatitis.

The foregoing and other features of the disclosure will become more apparent from the following detailed description, which proceeds with reference to the accompanying figures.

### BRIEF DESCRIPTION OF THE DRAWINGS

FIG. 1 is a schematic showing an exemplary breeding scheme for generating Oncopigs derived from diverse genetic backgrounds.

FIGS. 2A and 2B are representative images from Oncopig Cancer Model (OCM) fibrosis induction procedure. FIG. 2A is a celiac arteriogram showing conventional porcine hepatic arteries (arrows). FIG. 2B is a fluoroscopic image obtained after administration of ethanol and ethiodized oil showing deposition of radiopaque emulsion throughout the liver (arrows).

FIG. 3 is an image showing gross pathology of OCM liver 8 weeks after induction, demonstrating undulation and nodularity of hepatic capsular surface, as well as enhanced reticular pattern and discoloration (arrows) of hepatic parenchyma, consistent with macronodular fibrosis.

FIGS. 4A and 4B are representative images of Masson's trichrome stained Cohort 1 porcine liver sections histologically graded for fibrosis using porcine-adapted METAVIR scheme. FIG. 4A shows 8-week post-induction histology of a liver section showing METAVIR F3 fibrosis with marked expansion of portal areas and fibrous septa by abundant amounts of fibrosis (arrows), which extends into adjacent lobular parenchyma and surrounds and separates hepatocyte clusters. FIG. 4B shows histologically normal porcine control liver with normal pre-existing fibrous septa (arrows), which impart distinct pig liver lobular architecture. Images are 5× magnification.

FIGS. 5A and 5B are representative images of Masson's trichrome stained Cohort 2 porcine liver sections histologically graded for fibrosis using porcine -adapted METAVIR scheme. FIG. 5A shows a 2-week post-induction section demonstrating METAVIR F2 fibrosis, characterized by moderate fibrous expansion of portal areas and pre-existing septa (arrows). FIG. 5B shows a 20-week post-induction section with F2 fibrosis, evident by significant resolution of fibrosis and reestablishment of normal lobular architecture, with only mild fibrous expansion of portal areas and septa (arrows). Images are 5× magnification.

FIGS. 6A-6C are images showing liver mass following HCC liver engraftment in cirrhotic liver. FIG. 6A is an ultrasound image showing a hypoechoic 1 cm round intrahepatic mass (arrowhead). L=liver, GB=gall-bladder. FIG. 6B is a contrast-enhanced liver CT image depicting the same mass (arrowhead). FIG. 6C shows gross pathology of the resected mass.

FIGS. 7A-7C are images showing liver mass histology of the resected tumor mass from FIG. 6C. FIG. 7A shows a representative histologic section of resected liver mass revealing trabecular pattern of malignant cells consistent with HCC (H&E, 20× magnification). FIG. 7B is an image showing that the tumor contains wispy material (arrow) representing bile, typical of hepatocyte differentiation (H&E, 20× magnification). FIG. 7C is a representative image of non-tumorous liver adjacent to tumor nodule, demonstrating dense collagen bands (arrows) consistent with METAVIR grade 2-3 fibrosis (Masson's trichrome, 5× magnification).

FIG. 8 is a plot showing that Oncopig samples cluster by cell type based on transcriptomic profiles.

FIGS. 9A and 9B show that injection of AdCre in the pancreatic duct induces OCM pancreatic cancer tumors. FIG. 9A shows CT imaging one-year post-induction to evaluate pancreatic tumor formation (arrows). FIG. 9B shows gross histology of the pancreas shown in FIG. 9A.

FIGS. 10A-10C show that induced OCM pancreatic cancer tumor recapitulates a human pancreatic ductal adenocarcinoma. FIG. 10A is a series of H&E stained human pancreatic ductal adenocarcinoma (PDAC) samples, displaying varied tissue architecture with common features including ductal lesions with cellular atypia and a dense tumor stroma. FIG. 1OB is a series of H&E stained OCM pancreatic cancer tumors displaying histologic features consistent with human PDAC. FIG. IOC is a series of OCM pancreatic tumors stained for the pan-epithelial marker E-cadherin and the duct marker CK19, affirming a human PDAC histotype.

FIGS. 11A-1 ID show that OCM pancreatic cancer develops a tumor microenvironment analogous to human PDAC. The tumor microenvironments of human PDAC (FIG. 11A) and OCM pancreatic cancer (FIG. 1IB) display desmoplastic stroma as evidenced by Masson's trichrome staining. FIG. 11C shows positive staining of OCM pancreatic cancer tumor stroma for the mesenchymal marker Vimentin. FIG. 1ID shows dual staining of OCM pancreatic tumor with CK19 and aSMA to evaluate the presence of pancreatic stellate cells surrounding the tumors, which are considered the critical mediator of tumor-associated fibrosis.

FIGS. 12A-12D are graphs showing in vitro partitioning and conversion of 6-diazo-5-oxo-1-norleucine (DON) prodrugs to DON in Oncopig soft tissue sarcoma tumor cells vs. plasma. FIG. 12A demonstrates that when administering DON to cells in the supernatant (non-prodrug form), high levels are observed in the cell supernatant, with small amounts entering the cells. However, when delivered as a prodrug (FIGS. 12B-12D), the level of active compound (DON, y-axis) is much higher in the cells than the cell supernatant.

FIGS. 13A and 13B are graphs showing selective in vivo partitioning of DON to tumor cells (FIG. 13A) and DON prodrug (intact JHU 400) to plasma (FIG. 13B) in Oncopigs bearing sarcomas.

FIGS. 14A-14D are representative immunohistochemistry images of Oncopig sarcoma intratumoral CD3⁺ cells at 1O×(FIGS. 14A and 14B) and 40× magnification (FIGS. 14C and 14D).

FIGS. 15A-15F show the Oncopig sarcoma tumor microenvironment is infiltrated by perforin⁺ and granzyme B⁺ immune cells. Oncopigs were subcutaneously injected with AdCre and PBMCs and sarcoma tumors were harvested 7-21 days post-injection and analyzed by flow cytometry. FIGS. 15A-15C show perfbrhri cells as a percentage of live CD3⁺ gated cells (FIG. 15A), as a percentage of live CD3⁺CD4⁺ gated cells (FIG. 15B), and as a percentage of live CD3⁺ CD8β⁺ gated cells (FIG. 15C). *P<0.05. FIGS. 15D-15F show immunofluorescence in tumor tissue sections for CD3 (FIG. 15D), CD3 and CD8a (FIG. 15E), and granzyme B (FIG. 15F). DAPI was used as a nuclear counterstain for all immunofluorescence.

FIGS. 16A-16D are a series of graphs showing percentage of FoxP3+T cells in live CD3+ gated cells (FIG. 16A), percentage of FoxP3⁺ T cells in live CD4⁺CD8a⁻ gated T cells (FIG. 16B), percentage of FoxP3⁺ T cells in live CD4⁺CD8a⁺ gated T cells (FIG. 16C), and percentage of FoxP3+ T cells in live CD4 CD8a⁺ gated T cells (FIG. 16D) in peripheral blood and sarcoma tumor tissue from Oncopigs subcutaneously injected with AdCre and analyzed 7-21 days later.

FIGS. 17A and 17B show specific lysis of autologous tumor cells in vitro. Oncopigs were subcutaneously injected with AdCre and tumor cells and PBMCs were harvested 7-21 days after injection. FIG. 17A shows representative flow cytometric plots of control and tumor cells at 10 minutes (left) and 24 hours (right) post-coculture. FIG. 17B is a graph showing percentage specific killing of autologous tumor cells; data were normalized to adjust for cell turnover in no-effector cells control cultures. A titration of the effector target cell ratio is shown (*P<0.05 and **P<0.005).

FIGS. 18A-18E. FIG. 18A is a schematic representation of the Oncopig transgene showing gRNA target sites and primers used for PCR. IRES, Internal ribosome entry site. FIG. 18B shows KRAS′^{12D} and TP53 [symbol] editing efficiencies at multiple time points post transfection with Cas9 and gRNAs. FIG. 18C shows an alignment of frameshift mutations resulting in protein truncation for 2 Oncopig TP53^{R167H} HCC cell lines developed via single cell colony formation and screening (Reference, SEQ ID NO: 1; TP53 KO #1, SEQ ID NO: 2; TP53 KO #2, SEQ ID NO: 3) and schematic diagram of the mutations. Dashed line marks the cleavage position, and dashed grey boxes represent nucleotide deletions. Dotted regions represent frameshifts in predicted protein sequences. FIG. 18D shows positive arginase-1 staining (brown) of parental and TP53 [symbol] KO cell lines (scale bar, 300 pm). FIG. 18E shows cellular proliferation of Oncopig primary and TP53^{KI27″} KO HCC cell lines. Values represent mean±S.D. (n>3). **indicates P<0.001.

FIGS. 19A-19C illustrates most frequent INDELs detected by targeted Illumina sequencing 2 days post transfection for Oncopig HCC cells transfected with gRNA targeting KRAS [symbol] (FIG. 19A; SEQ ID NOs: 4-19) or TP53^{RI67H} (FIG. 19B; SEQ ID NOs: 20-35). Asterisks indicate unedited reads. Dashed line, cleavage position; red box, insertion; dashed grey box, deletion. FIG. 19C is sequence analysis for Oncopig HCC cells co-transfected with gRNAs targeting KRAS^{C12D} and TP53 [symbol] , confirming deletion of the region between the KRAS^{G12D} and TP53^{RI67H} transgenes (SEQ ID NO: 36).

FIGS. 20A-20C. FIG. 20A shows most frequent INDELs detected by targeted Illumina sequencing 2 days post transfection for Oncopig HCC cells transfected with gRNA targeting ARID1A (left; SEQ ID NOs: 37-68) or AXIN1 (right; SEQ ID NOs: 69-93). FIG. 20B illustrates ARID 1A KO mutations and resulting protein truncations for 3 of the 18 KO single cell clones that underwent phenotypic analysis (SEQ ID NOs: 94-98). FIG. 20C is a graph of cell proliferation results demonstrating increased cellular proliferation of all 3 ARID 1A KO compared to parental cell lines. **denotes p-value<0.001.

FIGS. 21A-21D show representative trichrome staining of liver biopsy performed 4 weeks post cirrhosis induction for Oncopig that underwent cirrhosis induction (FIG. 21A) and Oncopig that underwent cirrhosis induction plus alcohol feeding (FIG. 2IB), and representative trichrome staining of liver biopsy performed 8 weeks post cirrhosis induction for Oncopig that underwent cirrhosis induction (FIG. 21C) and Oncopig that underwent cirrhosis induction plus alcohol feeding (FIG. 2ID).

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is a reference TP53 nucleic acid sequence.

SEQ ID NOs: 2 and 3 are nucleic acid sequences of TP53 KO cell lines.

SEQ ID NO: 4 is a KRAS^{G12D} reference nucleic acid sequence.

SEQ ID NOs: 5-19 are exemplary INDELs from HCC cells transfected with gRNA targeting KRAS^{G12D}.

SEQ ID NO: 20 is a TP53^{KI77″} reference nucleic acid sequence.

SEQ ID NOs: 21-35 are exemplary INDELs from HCC cells transfected with gRNA targeting and ^{]]}53 [symbol] .

SEQ ID NO: 36 is a nucleic acid sequence from HCC cells co-transfected with gRNAs targeting KRAS^{G12D} and TP53^{r167H}.

SEQ ID NO: 37 is an ARID 1A reference nucleic acid sequence.

SEQ ID NOs: 38-68 are exemplary INDELs from HCC cells transfected with gRNA targeting AR1D1A.

SEQ ID NO: 69 is an AXIN1 reference nucleic acid sequence.

SEQ ID NOs: 70-93 are exemplary INDELs from HCC cells transfected with gRNA targeting AXIN1.

SEQ ID NO: 94 is an ARID1A reference nucleic acid sequence.

SEQ ID NOs: 95-98 are nucleic acid sequences of single cell clones with ARID1A KO mutations.

### DETAILED DESCRIPTION

Disclosed herein are animal models and processes that are applicable for diagnostic, drug, and device discoveries, as well as for testing of existing drugs and/or devices for new therapeutic applications or for use in combination therapies, referred to as “Modeling Oncology on Demand” (MOOD). MOOD is not restricted to the UI Oncopig (see, e.g., Schook et al., PLOS One 10:e0128864, 2015, referred to herein as the Oncopig), but is applicable to any Oncopig Cancer Model (OCM) or other pig model, that phenotypically reflects what is observed in humans clinically. The use of MOOD also permits trials to be conducted in the setting of relevant medical co-morbidities, including but not limited to liver cirrhosis, non-alcoholic steatohepatitis (NASH), obesity, cardiovascular disease, and other genetic and environmental predispositions. Due to the anatomical, physiological, and metabolic similarities between pigs and humans it also allows for the use of existing medical devices and clinical methodologies (e.g., lowering blood pressure or body temperature) during a therapeutic procedure.

Features of MOOD include: (1) the ability to create personalized tumors through genome editing in animals; (2) tissues and cells from defined tumors that support the use of existing clinical approaches and/or advanced radiologic imaging (e.g., ultrasound, CT, MRI, and PET), including the utilization of minimally invasive Interventional Radiology or surgical procedures; (3) the use of other biologies, drugs, or devices, or combining precise imaging guidance and targeted therapy; (4) creating defined microenvironments and co-morbidity conditions associated with genetically defined tumors; and (5) MOOD can support mono- or combination therapies by reducing the time and cost of clinical trials, decreasing the time to market by rapid cycling achievable in an animal model population, decreasing the number of false positive drugs at preclinical stages before progression to first-in-human or phase 1 clinical trials, enhancing the efficacy of preclinical animal testing, and/or reducing or eliminating human clinical trials on less promising drugs and devices.

### I. Terms

Unless otherwise noted, technical terms are used according to conventional usage. Definitions of common terms in molecular biology may be found in Lewin's Genes X, ed. Krebs el al, Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Redei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrated to other parts of the body, for example via the bloodstream or lymph system.

Co-morbid condition or co-morbidity: A disease, disorder, or condition that occurs simultaneously with a primary disease or disorder. A co-morbid condition may be independent of the primary disease or disorder, or may be caused by, or related to, the primary disease or disorder. In some examples herein, the primary disease or disorder is cancer (e.g., one or more tumors). Exemplary co-morbid conditions include liver cirrhosis, diabetes, obesity, hyperlipidemia, metabolic syndrome, cardiovascular disease, non-alcoholic fatty liver disease, non-alcoholic steatohepatitis, chronic kidney disease, and environmental or behavioral factors (e.g., high-fat diet, tobacco use, alcohol use, caffeine use, and marijuana use). Other exemplary co-morbid conditions include age (e.g., a young, adolescent, or senior subject), reduced functional or performance status, prescription and/or over-the-counter drug use, drug abuse or addiction, pregnancy, or hormone treatment (e.g., estrogen or testosterone treatment).

Control: A sample or standard used for comparison with an experimental sample, such as a sample from an untreated subject (such as a subject with the same condition as the experimental sample). In other embodiments, the control is a historical control or standard reference value or range of values (e.g., a previously tested control sample, such as a group of healthy subjects, or group of samples that represent baseline or normal values). Laboratory standards and values can be set based on a known or determined population value and can be supplied in the format of a graph or table that permits comparison of measured, experimentally determined values.

Subject: A living multi-cellular vertebrate organism, a category that includes human and non-human mammals. In one example, a subject is a human subject with a tumor or cancer.

Test compound: A “test compound” is any substance or any combination of substances that is useful for achieving an end or result. Any compound that has therapeutic potential (whether or not ultimately realized) can be tested using the methods of this disclosure. In particular examples, a test compound is a substance having chemotherapeutic potential.

Treating or ameliorating a condition: Reducing one or more signs or symptoms of a condition (such as a tumor or a co-morbidity) or symptom of a condition. Treatment of a condition can span the spectrum from partial inhibition (reduction) to substantially complete inhibition of the condition, symptom, or disease. In some examples, the term “ameliorating” refers to reducing or delaying the onset or progression of the condition or a symptom of a condition.

Tumor: The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant” or “cancer.” Neoplasia is one example of a proliferative disorder.

### II. Overview of Several Embodiments

The methods disclosed herein utilize an animal model of cancer, such as a porcine cancer model. In some examples, the methods utilize an inducible porcine cancer model, referred to as the “Oncopig” or “Oncopig Cancer Model” (OCM). This porcine model of cancer is a transgenic porcine line encoding Cre recombinase-inducible porcine transgenes encoding KRAS^{G12D} and TP53^{R167H} (see, e.g., Schook et al, PLOS One 10:e0128864, 2015, incorporated by reference herein). Cells from the Oncopig can be transformed in culture with an adenovirus encoding Cre (AdCre). Furthermore, injection of the transgenic pigs with AdCre results in formation of tumors.

The instant application discusses the disclosed methods in the context of the Oncopig. However, the disclosed methods are not limited to this model, and can be used with other porcine cancer models capable of recapitulating human cancers.

Disclosed herein are transgenic pigs with at least one cancer and at least one co-morbid condition, condition, wherein the pig is transgenic for Cre recombinase-inducible KRAS^{G12D} and TP53^{R167H}. In some non-limiting examples, the combination of at least one cancer and at least one co-morbid condition is not liver cancer (e.g., hepatocellular carcinoma) and any one of cirrhosis, obesity, and non-alcoholic steatohepatitis.

In some embodiments, disclosed herein are in vitro methods for screening for therapeutic compounds. The methods include contacting cells from a pig having at least one cancer and at least one co-morbid condition with one or more test compounds, measuring cell growth and/or survival, and identifying a compound as a therapeutic compound if the cell growth and/or survival is decreased compared to a control. In other embodiments, disclosed herein are in vivo methods for screening for therapeutic compounds. The methods include administering one or more test compounds to a pig having at least one cancer and at least one co-morbid condition, determining the effect of the one or more test compounds on the at least one cancer and/or the at least one co-morbid condition, and identifying a compound as a therapeutic compound if the at least one cancer and/or the at least one co-morbid condition is ameliorated compared to a control. In some examples, the pig is transgenic for Cre recombinase-inducible KRAS^{G12D} and TP53^{R167H}. In other examples, the methods include collecting cells from a pig that has one or more co-morbid condition, but does not have a tumor. The cells are then transformed in vitro and contacted with one or more test compounds. Cell growth and/or survival is measured and a compound is identified as a therapeutic compound if the cell growth and/or survival is decreased compared to a control. In some examples, a compound that is identified as a therapeutic compound is administered to a subject (such as a human subject) for treatment of the cancer and/or co-morbid condition.

In further embodiments, disclosed are methods of testing medical devices or procedures. The methods include administering one or more medical device or procedure to a pig having at least one cancer and at least one co-morbid condition, and determining the effect of the medical device or procedure on the at least one cancer and/or the at least one co-morbid condition compared to a control. In some examples, the pig is transgenic for Cre recombinase-inducible KRAS^{G12D} and TP53^{R167H}. In particular examples, medical device or procedure includes one or more of imaging, thermal therapy, targeted drug activation, targeted drug delivery, electroporation, and embolization (with or without a chemotherapeutic agent). In some examples, the medical device or procedure is administered to a subject (such as a human subject) for treatment of the cancer and/or co-morbid condition.

Also disclosed are methods of identifying one or more diagnostic or prognostic biomarkers, for example one or more biomarkers diagnostic or prognostic for at least one cancer and/or at least one co-morbid condition. In some examples, the methods include obtaining one or more samples from a pig having at least one cancer and at least one co-morbid condition, measuring levels of one or more biomarkers in the sample, and comparing the one or more biomarkers with a control. In some examples, the pig is transgenic for Cre recombinase-inducible KRAS^{G12D} and TP53^{R167H}.

In other embodiments, disclosed are methods of producing a personalized cancer model. The methods include obtaining a tumor sample from a subject (such as a subject with cancer, e.g., a human subject with cancer), sequencing the genome of tumor cells from the tumor sample, identifying one or more mutations in the genomic sequence of the tumor cells (e.g., one or more driver mutations), introducing the one or more mutations into isolated cells from a pig, to produce isolated pig cells comprising the one or more mutations, and administering the isolated pig cells comprising the one or more mutations to a pig to produce one or more tumors in the pig. In other examples, the method includes introducing the one or more mutations identified in the tumor cells directly in the pig (e.g., utilizing in vivo gene editing) to produce one or more tumors in the pig. In some examples, the pig has one or more co-morbid conditions. In particular examples, the pig is transgenic for Cre recombinase-inducible KRAS^{G12D} and TP53^{R167H}. In some examples, the methods include administering one or more treatments to the pig having one or more tumors, such as administering one or more test compounds (e.g., one or more cytotoxic or chemotherapeutic agents) and/or administering one or more medical device or procedure to the pig having one or more tumors. If the one or more therapeutic compound and/or one or more medical device or procedure ameliorates the cancer and/or the co-morbid condition, the one or more therapeutic compound and/or one or more medical device or procedure is administered to the subject.

### III. Modeling Cancer and Co-Morbidities

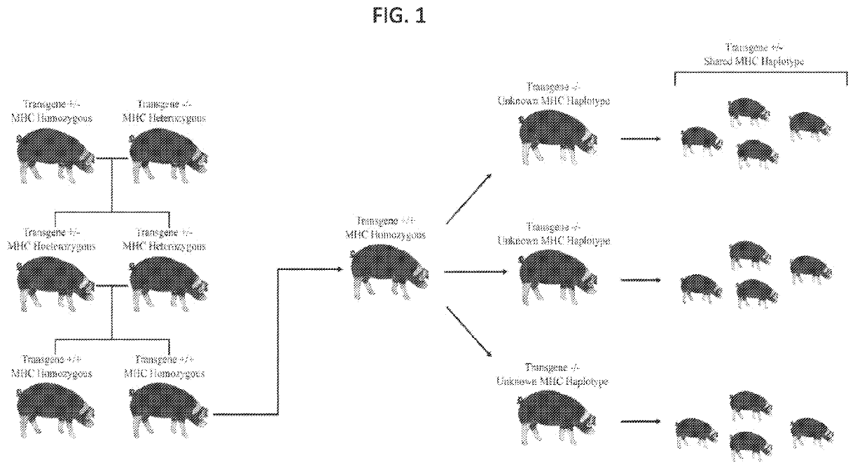

In some embodiments, the Oncopig is used as a model of cancer and one or more co-morbid conditions. An advantage of this model is that the Oncopig can be bred to a wide range of available pig breeds, allowing production of genetically diverse Oncopigs (e.g., FIG. 1), mimicking the diversity observed in clinical practice. This breeding scheme can be used to generate genetically diverse Oncopigs that are predisposed to a range of co-morbidities. In addition, as discussed below, one or more co-morbidities can be specifically induced in the Oncopig model, or in other pig breeds. Thus, the disclosed animals and methods can be used to model the effects of genetic background on disease development, progression, and treatment response.

As described in Example 2, below, a model of intrahepatic HCC tumors in cirrhotic liver has been developed in the Oncopig. This exemplary model utilizes alcohol-induced cirrhosis, which provides a significant improvement over current rodent models of cirrhosis, as no rodent model exists that fully mirrors human alcoholic liver disease by developing liver fibrotic stages via alcohol consumption.

As demonstrated with alcohol-induced cirrhosis, OCM can be used to model comorbid conditions, including treatment and/or reversibility of the conditions. This feature facilitates better predictability of the reversibility of comorbidities and their impact on cancer development, progression, and response to treatment. MOOD can be used in the development of treatments to reduce the likelihood of patients developing an irreversible comorbidity, and decrease the likelihood of a comorbidity to lead to cancer development. This concept can also be applied to model environmental or behavioral changes, including changes in diet, medication (prescription and/or over-the-counter), and recreational drug use or abuse, as well as conditions such as pregnancy, hormone therapy, age, and/or performance or functional status. This aspect of MOOD also facilitates identification of biomarkers that can be used to predict when a comorbidity has progressed to a chronic, irreversible form.

In some examples, the Oncopig has at least one cancer and at least one co-morbid condition. Exemplary cancers include liver cancer (e.g., hepatocellular carcinoma), sarcoma (e.g., soft tissue sarcoma), lung cancer, colorectal cancer, esophageal cancer, pancreatic cancer (e.g., pancreatic ductal adenocarcinoma), breast cancer, bladder cancer, kidney cancer, endometrial cancer, ovarian cancer, brain cancer, hematological cancer, or metastatic disease (e.g., metastatic liver disease). Exemplary co-morbid conditions include liver cirrhosis, diabetes, obesity, hyperlipidemia, metabolic syndrome, cardiovascular disease, non-alcoholic fatty liver disease, non-alcoholic steatohepatitis, chronic kidney disease, lung disease, cerebrovascular disease, peripheral vascular disease, high-fat diet, tobacco use, alcohol use, caffeine use, marijuana use, and recreational/illicit drug use. Tumors and co-morbid conditions are discussed below. Exemplary combinations of cancers and co-morbid conditions are provided in Table 1. These pigs can be used in the methods disclosed herein.

TABLE 1
Exemplary combinations of cancers and co-morbidities

| Cancer | Co-Morbidities |
|---|---|
| Hepatocellular carcinoma | Cirrhosis, NAFLD, NASH, Cardiovascular disease, hypertension, |
| | Diabetes. Obesity, metabolic syndrome, alcohol use., drug use |
| Sarcoma | Cardiovascular disease, hypertension, Diabetes, Obesity |
| Metastatic liver disease | Cirrhosis, NAFED, NASH, Cardiovascular disease, hypertension, |
| | Diabetes. Obesity, high-fat diet, metabolic syndrome, alcohol use, |
| | drug use |
| Lung cancer | chronic lung disease (e.g., COPD), tobacco use, Cardiovascular |
| | disease, hypertension, Diabetes, Obesity, |
| Colorectal cancer | Cardiovascular disease, hypertension, Diabetes. Obesity, |
| | metabolic syndrome, alcohol use, drug use, high-fat diet, tobacco |
| | use, caffeine use |
| Esophageal cancer | chronic lung disease (e.g., COPD), tobacco use, alcohol use, |
| | Cardiovascular disease, hypertension, Diabetes, Obesity, |
| Pancreatic cancer | Cardiovascular disease, hypertension, Diabetes. Obesity, |
| | metabolic syndrome, alcohol use, high-fat diet |
| Breast cancer | Alcohol use, Cardiovascular disease, hypertension, Diabetes, |
| | Obesity, high fat diet, pregnancy, post-partum |
| Bladder cancer | Chronic kidney disease, Cardiovascular disease, hypertension, |
| | Diabetes, Obesity |
| Kidney cancer | chronic kidney disease. Cardiovascular disease, hypertension, |
| | Diabetes, Obesity |
| Endometrial cancer | Cardiovascular disease, hypertension, Diabetes, Obesity, high fat |
| | diet, pregnancy, post-partum |
| Ovarian cancer | Cardiovascular disease, hypertension, Diabetes. Obesity, |
| | pregnancy, post-partum |
| Hematologic cancers | Cardiovascular disease, hypertension, Diabetes. Obesity, age |
| Neuroendocrine cancers | Diabetes, Obesity, high fat diet |

A. Cancers

In addition to HCC (discussed in Example 2) and sarcoma (see, e.g., Schachtschneider et al., Scientific Reports 7:2624, 2017), other cancers can be modeled utilizing OCM (or other porcine models). Non-limiting examples include metastatic disease (such as metastatic liver disease, metastatic lung disease, or metastatic bone disease), lung cancers, colorectal cancers, esophageal cancers, pancreatic cancers, breast cancers, bladder cancers, kidney cancers, endometrial cancers, ovarian cancers, neuroendocrine cancers, uterine cancers, brain cancers, and hematologic cancers. Exemplary cancers are discussed in more detail below.

In some embodiments, OCM is used to model metastatic liver disease. The liver represents an organ where many cancers, including breast, colorectal, esophageal, lung, melanoma, neuroendocrine, pancreatic, and stomach, metastasize. Metastatic spread to the liver also represents the predominant cause of mortality for many of these deadly cancers. Metastatic liver disease for the above-mentioned cancer types can be induced using a number of techniques. In some examples, cell lines representative of human metastatic liver diseases are autologously injected into Oncopigs (e.g., subcutaneously or intrahepatically) to produce tumors. In other examples, metastatic liver disease is produced through in vivo autochthonous development of primary tumors, followed by biopsy, dissociation to develop cell lines, followed by autologous injection to produce metastatic tumors. In another example, the biopsy samples are directly implanted into the liver (e.g., via ultrasound guided percutaneous injection). In a further example, cell lines are developed using one of the two above-described methods and an autologous suspension of cells is injected into the spleen for distribution into the liver as metastases or into a blood vessel for hematogenous spread to various sites and/or organ systems.

In other embodiments, OCM is used to produce models of lung cancer (e.g., lung adenocarcinoma, small cell lung cancer, and/or non-small cell lung cancer). In some examples, lung cancer is induced by delivering AdCre (such as aerosolized AdCre) into the trachea or by direct injection into the lung. In other examples, a nanoparticle harboring Cre recombination is introduced in the trachea (for example in an aerosolized form) or by direct injection of nanoparticles into the lung. In still further examples, lung cell types of interest are isolated and transformed in vitro. The transformed cells can then be injected into the lung, or can be injected subcutaneously, and the resulting tumor segments can be injected into the lung.

In further embodiments, OCM is used to develop models of colorectal cancer. In some examples, AdCre or a nanoparticle including Cre recombinase is delivered by colonoscope to target colon epithelial cells (e.g. , to produce colorectal adenocarcinoma). In other examples, a patch or gel including AdCre or a nanoparticle including Cre recombinase is placed in the colon to more specifically target tumor development. In still further examples, colon epithelial cells are isolated (for example, using laparoscopic techniques) and transformed in vitro. The transformed cells can then be injected into the lining of the colon, or can be injected subcutaneously, and the resulting tumor segments can be injected into the colon.

In additional embodiments, OCM is used to develop models of esophageal cancer (e.g., esophageal adenocarcinoma or squamous cell carcinoma). In some examples, AdCre or a nanoparticle including Cre recombinase is delivered by endoscopy to target esophageal epithelial cells. In other examples, a patch or gel including AdCre or a nanoparticle including Cre recombinase is placed in the esophagus to more specifically target tumor development. In still further examples, esophageal epithelial cells are isolated (for example, by esophagectomy) and transformed in vitro. The transformed cells can then be injected into the lining of the esophagus, or can be injected subcutaneously, and the resulting tumor segments can be injected into the esophagus.

In other embodiments, OCM is used to develop models of pancreatic cancer (e.g., pancreatic ductal adenocarcinoma) or neuroendocrine tumors. In some examples, AdCre or a nanoparticle including Cre recombinase is delivered into the pancreatic duct to target pancreatic ductal cells. An exemplary method of producing pancreatic ductal adenocarcinoma in the Oncopig is provided in Example 4. In other examples, pancreatic cells (such as pancreatic ductal cells) are isolated and transformed in vitro. The transformed cells can then be injected into the pancreas (for example, into the pancreatic duct), or can be injected subcutaneously, and the resulting tumor segments can be injected into the pancreas.

In other embodiments, OCM is used to develop models of breast cancer (e.g., invasive ductal carcinoma, invasive lobular carcinoma, medullary carcinoma, mucinous carcinoma, and/or papillary carcinoma). In some examples, AdCre or a nanoparticle including Cre recombinase is delivered into mammary tissue to target mammary gland cells. In other examples, mammary gland cells are isolated and transformed in vitro. The transformed cells can then be injected into mammary tissue, or can be injected subcutaneously, and the resulting tumor segments can be injected into the mammary gland.

In further embodiments, OCM is used to develop models of bladder cancer (e.g., urothelial bladder carcinoma). In some examples, AdCre or a nanoparticle including Cre recombinase is delivered via a urinary catheter to target bladder epithelial cells. In other examples, a patch or gel including AdCre or a nanoparticle including Cre recombinase is used on the inner lining of the bladder, to more specifically target tumor development. In other examples, bladder epithelial cells are isolated and transformed in vitro. The transformed cells can then be injected into the lining of the bladder, or can be injected subcutaneously, and the resulting tumor segments can be injected into the lining of the bladder.

In additional embodiments, OCM is used to develop models of kidney cancer (e.g., renal cell carcinoma). In some examples, AdCre or a nanoparticle including Cre recombinase is delivered via ureteroscopy to target renal cells (such as epithelial cells of the proximal tubule). In other examples, a patch or gel including AdCre or a nanoparticle including Cre recombinase is used to more specifically target tumor development to kidney epithelial cells (e.g., of the proximal tubule). In other examples, renal cells are isolated and transformed in vitro. The transformed cells can then be injected into the kidney, or can be injected subcutaneously, and the resulting tumor segments can be injected into the kidney.

In other embodiments, OCM is used to develop models of endometrial cancer. In some examples, AdCre or a nanoparticle including Cre recombinase is delivered to the endometrium (or uterus), for example, by hysteroscopy. In other examples, a patch or gel including AdCre or a nanoparticle including Cre recombinase is placed on the endometrium to more specifically target tumor development. In other examples, endometrial cells (such as endometrial lining epithelial cells) are isolated and transformed in vitro. The transformed cells can then be injected into the uterine lining, or can be injected subcutaneously, and the resulting tumor segments can be injected into the uterine lining.

In still further embodiments, OCM is used to develop models of ovarian cancer. In some examples, AdCre or a nanoparticle including Cre recombinase is delivered to the ovary, for example, by laparoscopy or direct injection in the ovary. In other examples, a patch or gel including AdCre or a nanoparticle including Cre recombinase is placed on the ovary to more specifically target tumor development. In other examples, ovarian cells are isolated and transformed in vitro. The transformed cells can then be injected into the ovary, or can be injected subcutaneously, and the resulting tumor segments can be injected into the ovary.

In additional embodiments, OCM is used to develop models of hematologic cancers (such as leukemia, lymphoma, or multiple myeloma). In some examples, AdCre or a nanoparticle including Cre recombinase is delivered to the bone marrow or lymph nodes to target specific subtypes (such as specific white blood cell subtypes). In other examples, specific cell types are isolated from bone marrow or lymph node tissue and transformed in vitro. The transformed cells are injected intravenously, directly into bone marrow, or to one or more lymph nodes.

B. Co-Morbid Conditions

The OCM cancer models described above can be utilized to further develop models that have one or more co-morbid conditions. In some embodiments, one or more co-morbid conditions are induced in the Oncopig. In other examples, the Oncopig is bred to a pig breed that is susceptible to one or more co-morbidities. An advantage of this model is that the Oncopig can be bred to a wide range of available pig breeds, allowing production of genetically diverse Oncopigs (e.g., FIG. 1), mimicking the diversity observed in clinical practice. This breeding scheme can be used to generate genetically diverse Oncopigs that are predisposed to a range of co-morbidities. Thus, the disclosed methods can be used to model the effects of genetic background on disease development, progression, and treatment response.

In some examples, the disclosed models of cancer and co-morbidities can be used to model the reversibility of co-morbid conditions and their impact on cancer development, progression, and response to treatment. In some examples, the Oncopig with one or more co-morbidities is treated and amelioration of the one or more co-morbidities is monitored. The effect of amelioration of the co-morbidity on the cancer (such as development and/or progression) is also monitored in some examples.

The concept of comorbidities described here expands beyond comorbid disease conditions to include any environmental condition, for example diet, activity level, additional drugs being taken for other conditions, or recreational drugs. In particular examples, and the effect of changes in these conditions, for example, changes in diet, medication, and/or recreational drug use on the co-morbidity and cancer are monitored.

Modeling of these comorbidities allows for measurement of the impact of different comorbid diseases on tumor development, progression, and treatment response, which allows for more predictive device, drug, and biomarker testing. Examples and methods for producing cancer and co-morbidity models using the Oncopig are described in detail below.

In some embodiments, the co-morbidity is liver cirrhosis. Exemplary methods of inducing cirrhosis, alone or in combination with HCC, are provided in Examples 1 and 2. In other examples, cirrhosis can be induced by administering a combination of ethanol and lipopolysaccharide to the pig (e.g., ethanol:LPS:ethiodized oil), such as by injection into the hepatic arterial circulation.

In other embodiments, the co-morbidity is diabetes mellitus. Transgenic pig models that develop diabetes have previously been developed. Exemplary transgenic pig models of diabetes include glucose-dependent insulinotropic polypeptide (GIP) dominant-negative transgenic pigs (e.g., Renner et al., Diabetes 59:1228-1238, 2010; Renner et al, Diabetes 61:2166-2175, 2012), insulin C94Y transgenic pigs (e.g., Reimer et al, Diabetes 62: 1505-1511, 2013), and hepatocyte nuclear factor 1a dominant-negative transgenic pigs (e.g., Umeyama etal, J. Diabetes Complications 31:796-803, 2017). These pigs are bred with the Oncopig to develop diabetic pigs capable of developing tumors.

In other examples, the Oncopig is bred with obese pigs (e.g, Ossabaw pigs) to produce a model with an important risk factor for diabetes, as well as being capable of developing tumors. In other examples, diabetes is chemically induced in the Oncopig. Methods of inducing diabetes in pigs are known, and include administering alloxan in combination with high fat diet (Yucatan and Sinclair minipigs), nicotinamide and SZT or alloxan (Gottingen minipigs), SZT (Yorkshire pigs). See, e.g., Bellinger et al, ILAR Journal 47:243-258, 2006; incorporated herein by reference in its entirety. Thus, in some examples, the methods include cross-breeding Oncopigs with these pig lines to produce pigs capable of developing tumors and chemically induced diabetes.

In further embodiments, the co-morbidity is obesity, hyperlipidemia, and/or metabolic syndrome. In some examples, the Oncopig is crossbred with a pig breed that is predisposed to obesity, for example, when fed a high-fat diet. Exemplary pigs that are predisposed to obesity include Gottingen minipigs, which serve as a model for metabolic syndrome when fed a high fat diet. In other examples, Yucatan minipigs develop obesity when overfed a diet high in saturated fats and carbohydrates with a high glycemic index. Ossabaw pigs are a model for metabolic syndrome (obesity, insulin resistance, glucose intolerance, dyslipidemia, and hypertension). When these pigs are fed a high-fat and high-cholesterol diet, they are more obese, have higher peak glucose and insulin following glucose tolerance testing, have increased serum triglycerides, and increased blood pressure compared to controls. Thus, in some examples, a model of cancer and obesity (or related conditions) is produced by breeding Oncopigs with Gottingen minipigs, Yucatan minipigs, or Ossabaw pigs, and feeding a high-fat diet. In other examples, a model of cancer and obesity (or related conditions) is produced by breeding Oncopigs with phenotypically diverse breeds and feeding a high-fat diet.

In further embodiments, the co-morbidity is cardiovascular disease. In some examples, the Oncopig is crossbred with a pig breed that is predisposed to cardiovascular disease. A number of minipig breeds develop coronary, aortic, iliac, and carotid atherosclerotic lesions (anatomical locations extremely relevant to the human condition), when fed a high-fat high cholesterol diet. The pig can develop spontaneous lesions in the vasculature and cardiac valves, and has been widely used to study atherosclerosis. In one non-limiting example, the porcine model is the Ossabaw pig (e.g., Etherton et al, Lipids 15:823-829, 1980; Neeb et al, Comp. Med. 60:300-315, 2010; Dyson et al, Comp. Med. 56:35-45, 2006; Tumbleson and Schook, Swine in Biomedical Research, Vol. 1-2, New York, N.Y., Plenum Publishing, 1996). A porcine model of early aortic valve sclerosis has also been developed through feeding a high fat/high cholesterol diet (e.g., Yorkshire swine, Sider el al, Cardiovasc. Pathol. 23:289-297, 2014). In other examples, cardiovascular disease can be induced in the Oncopig. For example, atherosclerosis can be induced in pigs through an atherogenic diet. In addition, pig models of atherosclerotic coronary heart disease and heart failure can be produced through surgically constricting the coronary arteries or artificially producing intracoronary embolisms. Thus, a cancer model with cardiovascular co-morbidity is produced by breeding Oncopigs with a pig breed predisposed to cardiovascular disease (and feeding high-fat diet, in some cases), by inducing cardiovascular disease in the Oncopig, or a combination thereof.

In other embodiments, the co-morbidity is non-alcoholic fatty liver disease (NAFLD) and/or non-alcoholic steatohepatitis (NASH). In some examples, the Oncopig is crossed with a pig breed that is predisposed to NAFLD and/or NASH. In one example, the breed is Ossabaw swine, which when fed a “Western” or “NASH diet” develop severe metabolic syndrome with a markedly abnormal liver histology that mimics human NAFLD and NASH. When Ossabaw pigs are fed a high fat NASH diet, they develop metabolic syndrome and progressive histologic features of NASH including extensive hepatocyte ballooning and fibrosis as well as progressive Kupffer cell accumulation with vacuolization (e.g., Lee et al, Hepatology 50:56-67, 2009). Thus, in some examples, a cancer model with NAFLD and/or NASH co-morbidity is produced by crossing Oncopigs with Ossabaw pigs and feeding the resulting pigs with a “Western” or “NASH” diet. This approach can be applied to any number of obesity prone pig breeds (including but not limited to Gottingen minipigs and Yucatan pig breeds), as well as pregnant and neonatal pigs.

In another embodiment, the co-morbidity is chronic kidney disease. In some examples, a cancer model with chronic kidney disease is produced by inducing chronic kidney disease in the Oncopig. Chronic kidney disease can be induced in pigs through bilateral renal artery stenosis and diet-induced dyslipidemia (for example, by feeding a high-fat high-cholesterol diet).

In yet further embodiments, the “co-morbidity” is diet, such as high-fat, high-sugar, and/or high-cholesterol diet (e.g, “Western” diet), “Mediterranean” diet, vegetarian diet, or vegan diet. The impact of diet on cancer is modeled by feeding the Oncopig a selected diet.

In other embodiments, the “co-morbidity” is the effect of drugs (including prescription, non-prescription (e.g., over-the-counter), and “recreational” drugs or drugs of abuse) on cancer development, progression, and response. In some examples, the Oncopig is administered one or more drugs (including, but not limited to tobacco, alcohol, caffeine, and/or marijuana) and effects on cancer development, progression, and/or response are monitored over time.

### IV. Therapeutic Development and Testing

In some embodiments, OCM is used as a model for identifying potential therapeutic compounds, testing the efficacy and/or dosage of therapeutics, and/or pharmacokinetic analyses in cancer (alone or in the presence of co-morbidities). The identified therapeutic compounds are then administered to human subjects, e.g., in a clinical trial and/or treatment setting.

In some embodiments, the methods include contacting cells from an OCM pig having at least one cancer and at least one co-morbid condition with one or more test compounds, measuring cell growth and/or survival, and identifying a compound as a therapeutic compound if the cell growth and/or survival is decreased compared to a control. In other embodiments, the methods include administering one or more test compounds to an OCM pig having at least one cancer and at least one co-morbid condition, determining the effect of the one or more test compounds on the at least one cancer and/or the at least one co-morbid condition, and identifying a compound as a therapeutic compound if the at least one cancer and/or the at least one co-morbid condition is ameliorated compared to a control.

In other examples, the methods include collecting cells from a pig that has one or more co-morbid condition, but does not have a tumor. The cells are then transformed in vitro and contacted with one or more test compounds. Cell growth and/or survival is measured and a compound is identified as a therapeutic compound if the cell growth and/or survival is decreased compared to a control.

In further embodiments, disclosed are methods of testing medical devices or procedures. The methods include administering one or more medical device or procedure to a pig having at least one cancer and at least one co-morbid condition, and determining the effect of the medical device or procedure on the at least one cancer and/or the at least one co-morbid condition compared to a control. In some examples, the pig is also administered one or more test compounds (e.g., one or more therapeutic compounds), in order to test combinations of therapeutic agents (such as chemotherapeutic agents) and medical devices and/or procedures.

Parameters that are measured are selected based on the test compound, medical device, and/or procedure being tested as well as the cancer and co-morbid condition(s) present in the test pig. Exemplary parameters include blood, serum, or plasma levels for one or more compounds of interest, such as tumor marker(s) (for example, CA19-9, CA-125, CEA, or AFP), immune cell populations, circulating tumor cells, lipids (for example, cholesterol, triglycerides, LDL, or HDL), liver function markers (for example, ALT or AST), and/or kidney function markers (for example, creatinine or BUN). In other examples, the parameters include urine metabolites. When a medical device or procedure is being tested, additional parameters measured may include technical success, clinical success, adverse events, imaging response, pathologic response, biomarker response, symptom amelioration, time-to-progression, progression-free survival, overall survival, and quality of life.

In some embodiments, the methods disclosed herein include measuring the effect of a test compound (such as a therapeutic agent, for example, a chemotherapeutic agent) and/or a medical device or procedure and determining if the effect is altered compared to a control. The control can be any suitable control against which to compare the effect of the compound and/or other intervention. In some embodiments, the control is a subject with the same condition (e.g., the same cancer and co-morbidity) that is untreated or treated with a control substance or intervention. In other examples, the control is a healthy subject. In some embodiments, the control is a reference value (e.g., normal range for pigs). For example, the reference value can be derived from average values obtained from a group of subjects having the same condition(s) and/or a group of healthy subjects.

A. Therapeutic Compounds

The Oncopig model, including pigs with one or more cancers and one or more co-morbidities are useful for screening for therapeutic compounds (for example, for efficacy and/or dosing). OCM and human HCC exhibit similar responses to chemotherapeutics (Example 8), indicating that OCM is a suitable platform for identifying compounds with therapeutic activity for further testing, thereby increasing the likelihood of successful clinical trials in humans.

Failures in all phases of clinical trials have increased over the past few decades, with a substantial portion occurring for safety reasons. One of the key areas for which improvements are required is in the screening for drugs likely to fail clinical trials. OCM provides the ability to define “effective drug” dosages and move away from the concept that the maximum tolerated dose is the best dose. Conducting trials using this method will facilitate identification of new candidate therapeutics and translation of optimal effective drug dosages not possible with murine models due to their differential metabolic rate compared to humans. This can shift current practice away from dosage screenings based solely on toxicity (maximum tolerated dose) in phase 1 clinical trials, providing a practical and predictable model for more effective advancement to clinical trials. This is particularly important for combination therapies utilizing multiple drugs where there is a need to define effective dosage combinations and optimal administration sequence, rather than the maximum tolerated dose for each compound.

Exemplary test compounds include, but are not limited to, peptides, such as soluble peptides, including but not limited to members of random peptide libraries, and combinatorial chemistry-derived molecular libraries made of D-and/or L-configuration amino acids, phosphopeptides (including, but not limited to, members of random or partially degenerate, directed phosphopeptide libraries), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as so-called natural products or members of chemical combinatorial libraries), molecular complexes (such as protein complexes), or nucleic acids (including, but not limited to, miRNA, siRNA, or antisense compounds).

Appropriate compounds can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds. In other examples, test compounds include compounds that have previously been identified as having, or potentially having, therapeutic effects, for example, in in vitro or animals (e.g., rodent, canine, or non-human primates).

Additional test compounds include chemotherapeutic or cytotoxic agents (or combinations thereof). Exemplary agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and thioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, atezolizumab, avelumab, ipilimumab, ofatumumab, nivolumab, pembrolizumab, rituximab, durvalumab, and trastuzumab; photosensitizers, such as aminolevulinic acid, methyl aminolevulinate, porfimer sodium, and verteporfm; and other agents, such as alitretinoin, altretamine, amsacrine, anagrelide, arsenic trioxide, asparaginase, bexarotene, bortezomib, celecoxib, denileukin diftitox, erlotinib, estramustine, gefitinib, hydroxycarbamide, imatinib, pentostatin, masoprocol, mitotane, pegaspargase, and tretinoin. Additional agents include other immunotherapies, such as sipuleucel-T, tisagenlecleucel, axicabtagene ciloleucel, and talimogene laherparepvec.

Pigs are an important preclinical toxicology model. Thus, in additional embodiments, disclosed herein are methods of pharmacokinetic analysis using the Oncopig with one or more cancers and one or more co-morbidities. The methods include administering one or more compounds to a pig with at least one cancer and at least one co-morbid conditions and measuring one or more pharmacokinetic parameters. In some examples, the pharmacokinetic parameters include drug partitioning (e.g., partitioning to plasma or cells, such as tumor cells), prodrug conversion to active compound(s), plasma concentration (e.g., maximum concentration (Cmax)), time to reach Cmax (tmax), elimination half-life (ti/2), and clearance.

B. Medical Devices or Procedures

Disclosed herein are methods of testing an interventional treatment, such as a medical device or procedure, including treating a pig having at least one cancer and at least one co-morbid condition with one or more medical device or procedure and determining the effect of the medical device or procedure on the at least one cancer and/or the at least one co-morbid condition compared to a control. In some examples, the intervention, such as using a medical device or procedure includes one or more of imaging, thermal therapy, targeted drug activation, targeted drug delivery, electroporation, and embolization (with or without a chemotherapeutic agent). The medical device or procedure can in some examples be tested in combination with one or more therapeutic agents (such as one or more chemotherapeutic agents).

For many cancers, thermal therapy is considered an important minimally invasive technique to destroy cancer tissue. Thus, in some examples, the medical device or procedure is a thermal therapy, such as cryoablation, radiofrequency ablation, microwave ablation (MWA), electroporation, and/or high-intensity focused ultrasound (HIFU). In addition, drug activation and targeting of drug delivery in vivo is becoming increasingly important due to the toxicities associated with many systemic treatments that show promise in preclinical settings. Thus, in some examples, the medical device or procedure includes use of laser, radiation, or ultrasound energy, or any other activation catalyst to allow for targeted activation of a given drug/treatment. In other examples, the medical device or procedure is targeted drug delivery approaches, for example using microbubbles, nanoparticles, or magnetization. In another example, the medical device or procedure includes chemoembolization. In further examples, the methods include image-guided localized drug delivery.

Specifically with regard to liver cancer (HCC), many recent studies have demonstrated the efficacy of interstitial ablative approaches including chemical and thermal ablation. Despite promising results, current systems remain highly operator dependent, and cannot treat many tumors due to insufficient control of the size and shape of the treatment zone, and limited control over ablator trajectory within tissue. Remedying these problems requires advances in delivery device design, robust image guidance, and precise steering of the ablator device to the desired target location with conformal energy delivery. A large animal cancer-comorbidity model paralleling human size and anatomy (as the OCM does) provides a beneficial platform to configure, test, revise, standardize, and/or optimize therapeutic devices (e.g., for cancer treatment) with assessment of impact on normal or diseased tissue.

As it pertains to liver cancer as proof of concept, while currently available HCC cell lines (e.g. HepG2) allow in vitro therapeutic screening, they do not permit in vivo assessment given inability to grow as viable solid tumors. Although the VX2 carcinoma is amenable to in vivo testing, bench top testing is precluded by lack of an accessible VX2 cell line. The OCM is the only available platform that permits in vitro therapeutic screening using HCC cell lines with the ability to translate promising strategies to in vivo investigation by means of solid intra-hepatic tumors. The OCM serves as an optimal platform to configure, test, revise, and optimize therapeutic combinations of devices and drugs for cancer treatment.

In further examples, the medical device or procedure includes imaging methods, such as computed tomography (CT), magnetic resonance imaging (MRI), and/or positron emission tomography (PET) radiologic technology. The OCM model is amenable to developing and establishing medical imaging standards related to diagnosing tumors and tracking treatment response using accepted radiologic criteria, a critical facet of therapeutic discovery and validation. Moreover, vascular and tissue enhancement in the pig are similar to that obtainable in patients at injection volumes and rates typically used in clinical practice (Gierada etal., Radiology 1999; 210:829-834), and PET radiotracers accumulate in porcine tissues in a manner comparable to that observed clinically (Piert et al, Eur J Nucl Med 1999; 26:95-109), allowing use of a cancer-comorbidity pig to be used as a platform for development, testing, and optimization of new contrast agents.

### V. Biomarker Identification

In further embodiments, the OCM is used to identify biomarkers (e.g., for diagnostic and/or prognostic tests). Due to the many physiological, anatomical, immunological, genetic, and epigenetic similarities with pigs and humans, biomarkers identified in these models have a high probability of leading to useful biomarkers for predictive and diagnostic tests in humans. These biomarkers can be developed through use of a cell or of a population of cells derived from an animal that models an oncological human disease and a secondary comorbid condition, for the in vitro or ex vivo identification of a cancer/comorbid microenvironment specific biomarker usable to diagnose a cancer, comorbidity, or to predict the optimal compound for treating a cancer. Inducible cancer models allow for knowledge of “time zero,” the time at which a tumor initially forms. This information allows for profiling of animal pre- and post-tumor development, which can facilitate identification of early detection biomarkers.

Thus, in some examples, the methods include obtaining one or more samples from a pig having at least one cancer and at least one co-morbid condition, measuring levels of one or more biomarkers in the sample, and comparing the one or more biomarkers with a control (e.g., a pig that does not have the at least one cancer and/or the at least one co-morbid condition). The sample may include one or more of blood, serum, plasma, tissues (e.g., tissue sample or biopsy, ear notch or tail sample, or hoof), saliva, urine, feces, tumor biopsy, tumor cells, cell-free tumor DNA, and/or circulating tumor cells. In other examples, the sample includes cells isolated from the pig, and in some examples, grown in vitro. In some examples, biomarkers include gene expression, DNA methylation and other epigenetic changes, protein concentrations, metabolites, and microbiome compositions. The one or more biomarkers are correlated with one or more conditions, such as presence or stage of the cancer and/or co-morbidity, for example, to identify diagnostic and/or prognostic biomarkers for the one or more cancer or co-morbid condition.

Various combinations of tumors, driver mutational profdes, and comorbidities are used for investigation of biomarkers associated with specific tumor/comorbidity combinations, to identify biomarkers that can lead to predictive tests in human, facilitating more accurate detection and diagnosis of tumors, comorbidities, and specific combinations in patients. Finally, biomarkers associated with therapeutic outcomes for different combinations of tumors/comorbidities can be identified to improve treatment stratification and patient prognosis in clinical practice.

### VI. Personalized Tumor Model

In yet further embodiments, pigs (such as OCM) are used as a personalized tumor model. Utilizing these approaches, personalized tumor models are developed to facilitate performance of a trial on a patient's tumor. Through a combination of tumor and comorbidity induction as described above, animal cohorts representative of individual patients are developed to screen for optimal treatment strategies for that individual.

Thus, in some embodiments, a tumor sample (e.g., a tumor biopsy) is obtained from a human subject. Nucleic acids (such as genomic DNA or RNA) from the tumor sample are sequenced to identify one or more mutations (such as one or more driver or passenger mutations) in the tumor. The one or more mutations are introduced into pig cells (such as OCM cells or normal pig cells) in vitro or in vivo, for example using genetic editing techniques. In one non-limiting example, the mutation(s) are introduced into the cells using a CRISPR-Cas9 system. An exemplary CRISPR-Cas9 system is provided in Example 7. However, any CRISPR or other genetic editing system can be used. The pig cells may be tumor cells (such as Oncopig cells exposed to Cre recombinase to induce transgene expression) or noncancerous (e.g., primary) cells. Introduction of the mutation(s) into primary cells will render them tumorigenic.

In embodiments where the one or more mutations are introduced into pig cells in vitro, the isolated cells including the introduced mutation(s) are administered to a pig, which may be an Oncopig, or a non-transgenic pig. In some examples, the cells are injected into the pig subcutaneously, followed by engraftment of the resulting tumor into the relevant organ. In other examples, the cells are injected directly into the relevant organ. In other examples, the cells are administered systemically, for example to model metastatic disease. In some examples, the pig also has or is induced to have one or more co-morbidities (e.g., one or more co-morbidities that are present in the subject from which the tumor sample was obtained). The pig is then maintained for a sufficient period of time for development of tumors that are “personalized” to the subject. In other embodiments where the one or more mutations are introduced into pig cells in vitro, the cells may be maintained in vitro and used to test one or more therapeutic compounds in vitro, for example, before, concurrently, or after injection into a pig.

In other embodiments, the one or more mutations is introduced into pig cells in vivo. In such examples, the one or more mutations are introduced into pig cells using in vivo gene editing approaches (such as CRISPR). The pig may be an Oncopig, or a non-transgenic pig. In some examples, the pig also has or is induced to have one or more co-morbidities (e.g., one or more co-morbidities that are present in the subject from which the tumor sample was obtained). The pig is then maintained for a sufficient period of time for development of tumors that are “personalized” to the subject

Following formation of the “personalized” tumor in the pig, one or more therapeutic interventions are tested. In some examples, one or more therapeutic interventions (including one or more therapeutic compounds, devices, procedures, or any combination thereof) are tested in vivo. In other examples, tumor cells are isolated from the “personalized” tumor and one or more therapeutic compounds are tested in vitro. Response to treatment is compared to control subjects (e.g., untreated pigs with the same personalized tumor) and/or standard-of-care treatments to identify treatment strategies for the human subject. In some examples, the therapy that produced the most complete response in the pig would be translated to the human subject. In other examples (e.g., a co-clinical trial), a cohort of pig subjects are developed and bred to be tested alongside an active clinical trial. This can help eliminate the accrual barrier that often is a limiting factor in the success of clinical trials.

### EXAMPLES

The following examples are provided to illustrate certain particular features and/or embodiments. These examples should not be construed to limit the disclosure to the particular features or embodiments described.

### Example 1

### Development of Co-Morbid Liver Cirrhosis in Model of Hepatocellular Carcinoma

At least portions of this example were published in Gaba el al, J. Vase. Interv. Radiol. 29: 1194-1202, Epub Jun. 7, 2018; which is incorporated herein by reference in its entirety.

The prevalence of liver cirrhosis approximates 4.5-9.5%, affecting hundreds of millions of people worldwide and more than 600,000 in the United States. This disease accounts for approximately 2% of all global mortality, approximating 1 million deaths per year. Among causes of liver cirrhosis, alcoholic liver disease underlies approximately 20% of deaths. Liver cirrhosis increases the risk for development of HCC, an aggressive malignancy that spans more than 780,000 new diagnoses and 750,000 annual deaths. These staggering data highlight the urgent need for further investigation into HCC detection, development and natural history, and response to LRT in its native comorbid cirrhotic background.

As the health status of the liver can also have profound effects on HCC tumor biology, treatment allocation, and response to therapy, a large animal model capable of exhibiting both HCC and liver cirrhosis concurrently is a valuable resource for advancing preclinical investigation of HCC detection, development, natural history, and response to treatment in its native comorbid cirrhotic background. We therefore utilized the innovative OCM to develop alcohol induced fibrosis in a porcine model capable of developing HCC tumors. The ability to concurrently induce liver cirrhosis and HCC in the OCM provides the opportunity to assess the role of chronic liver disease in HCC tumorigenesis.

### Methods

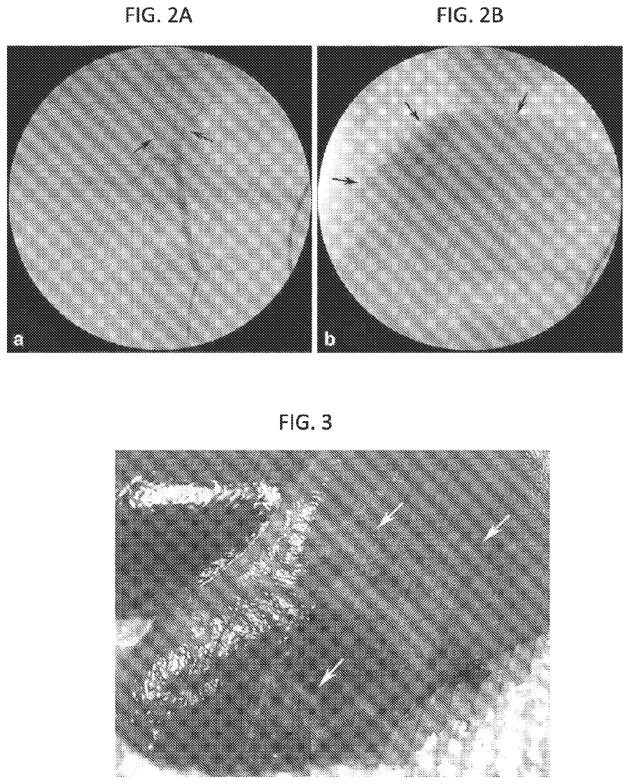

Fibrosis induction: Fibrosis inductions were performed for 10 Oncopigs. All fibrosis induction procedures were performed by one of two board-certified Interventional Radiology (IR) physicians according to a modification of the methodology described by Avritscher et al., J. Vase. Interv. Radiol. 22: 1329-1334, 2011. At 8-weeks of age, Oncopigs underwent anesthetic induction, followed by intubation and maintenance with 1-3% isoflurane. Angiography was performed using a C-arm (OEC Medical Systems series 9600; GE Healthcare, United Kingdom). With the animal supine, the groin was sterilely prepared. Ultrasound-guided vascular access was gained via the common femoral artery with placement of a 5 French sheath (Pinnacle; Terumo Medical Corporation, Somerset N.J.). Using standard catheter and wire techniques, celiac arteriography was performed using a 5 French catheter (Sos Omni Selective; AngioDynamics, Latham N.Y.) (FIGS. 2A and 2B).

A coaxial 3 French microcatheter (Renegade Hi-flo; Boston Scientific, Marlborough Mass.) was advanced into the common hepatic artery using a micro-guidewire. Arteriography was performed with iohexol (Omnipaque-300; Amersham Health, Princeton N.J.). The microcatheter was then advanced into the proper hepatic artery, and 0.75 mL/kg of a 1:3 v/v emulsified mixture of absolute ethanol and ethiodized oil (Lipiodol; Guerbet, Villepinte France, used to deliver ethanol into the liver and transiently embolize the liver microcirculation) was slowly infused by hand injection over 30 minutes. Dosing of the administered ethanol and ethiodized oil emulsion was derived by dividing the 28 mL maximally tolerated dose by mean pig weight reported in the study of Avritscher et al., 2011. Upon completion of the infusion, all devices were removed, and hemostasis was achieved via manual compression.

Clinical and laboratory assessment: OCM subjects in Cohort 2 underwent clinical assessment at baseline and biweekly post-induction thereafter, which included evaluation for ascites via ultrasound, as well as examination for the presence of hepatic encephalopathy (HE) resulting in neurologic impairment. Clinical assessments were performed by a board-certified laboratory animal veterinarian and a medical student research associate who were not blinded to treatment. HE was determined by neurological assessment of general appearance (quiet versus bright, alert, and responsive), cooperativity, appetite, and gait instability, as well as through a leg placement test. OCM subjects in both cohorts underwent laboratory testing at baseline and biweekly post-induction thereafter, which included standard blood chemistries relevant to the diagnosis and staging of chronic liver disease and pertinent for patient eligibility for locoregional therapy (LRT) for liver cancer. Laboratory evaluations included a complete blood count, total bilirubin, alkaline phosphatase, aspartate aminotransferase (AST), albumin, and prothrombin time. This assessment is consistent with components of the Child-Pugh score, which is used to assess chronic liver disease in humans. Laboratory testing for healthy control Oncopigs was performed at age-matched time points equivalent to 2-, 4-, 6-, and 8-weeks post-induction.

Measurement of portal hypertension: Portal hypertension is a sequela of liver fibrosis/cirrhosis, and the described fibrosis/cirrhosis induction method has been shown to result in measurable elevation of the hepatic venous pressure gradient (see Avritscher et al., 2011), allowing for use of the model for evaluation of portal hypertension related pharmaceuticals and interventions. For measurement of portal hypertension, a balloon-occlusion catheter is advanced under fluoroscopy via a right internal jugular or common femoral venous approach into a hepatic vein. The catheter is then used to measure free and wedged hepatic venous pressures, and the hepatic venous pressure gradient (HVPG).

Liver biopsy: Liver biopsies were obtained every 2 weeks for 20 weeks post fibrosis induction for 5 of the induced Oncopigs (Cohort 2). Percutaneous ultrasound-guided liver biopsy procedures were performed by a board-certified IR physician, a board-certified laboratory animal veterinarian, or a medical student research associate under the direct supervision of one of these practitioners. Liver biopsies were undertaken in a surgical suite following animal subject intubation and maintenance under general anesthesia. With the animal supine, the abdomen was sterilely prepared. An 18-gauge automated biopsy device (BioPince; Argon Medical Devices, Plano Tex.) was then advanced into the right liver lobe (selected given favorable subcostal sonographic visibility), and three to four 2-cm long core specimens were sequentially obtained and transferred to a container containing 10% neutral buffered formalin for fixation and pathological processing.

Liver tissue harvest: Euthanasia was performed at 8-weeks post induction (Cohort 1; n=5) and 20-weeks post induction (Cohort 2; n=5). Healthy control subjects (n=5) were sacrificed at age-matched time points consistent with Cohort 1. Necropsy was performed and Oncopig livers were harvested in their entirety. Next, representative samples of liver tissue weighing approximately 20 g were taken from both fibrotic and normal appearing liver areas, and transferred to a container containing 10% neutral buffered formalin for fixation and pathological processing.

Histological evaluations: Formalin fixed liver samples were embedded in paraffin, sectioned at 4-micron thickness, and mounted onto glass slides. Slides were stained using H&E and Masson's trichrome. Descriptive and semi-quantitative histopathological analyses were performed by a board-certified veterinary pathologist blinded to the treatment arm. For analysis, whole slides were scanned using a Hamamatsu Nanozoomer scanner (Hamamatsu Photonics, Hamamatsu Japan), and digital images were visualized with NDP.view2 software (Hamamatsu) and graded for fibrosis and inflammation according to a porcine-adapted METAVIR system (Tables 2 and 3) developed to provide inflammation and fibrosis scores comparable to the clinically employed METAVIR system used to diagnose liver cirrhosis in clinical patients. In addition, digital images of trichrome stained slides were also imported to ImageJ (NIH) using BioFormats, and subjected to color deconvolution for quantification of trichrome positive collagen, expressed as a percentage of total liver tissue section area.

TABLE 2
Porcine-adapted modified METAVIR fibrosis grading scheme

| Grade | Description |
|---|---|
| F0 | Normal porcine liver no increase in fibrosis |
| F1 | Mild Fibrous expansion of portal areas and/or mild thickening/ |
| | expansion of few random segments of normal pre-existing |
| | fibrous septa |
| F2 | Mild to moderate fibrous expansion of portal tracts and multiple, |
| | random, noncontiguous segments of normal fibrous septa |
| | surrounding multiple hepatic lobules ± presence of thin bands |
| | of fibrosis extending from septa or portal tracts into |
| | adjacent lobular parenchyma |
| F3 | Moderate to marked fibrous expansion of contiguous segments |
| | of fibrous septa surrounding multiple hepatic lobules; fibrous |
| | expansion, can involve contiguous segments of septa, and |
| | partially encircle hepatic lobules, but typically does not |
| | completely circumscribe lobules. Presence of fibrous connective |
| | tissue which dissects into lobular parenchyma, surrounding and |
| | separating cords of hepatocytes |
| F4 | Cirrhosis; normal fibrous septa surrounding hepatic lobules are |
| | expanded by moderate to marked amounts of fibrous connective |
| | tissue, with some portal bridging, and frequent dissection into |
| | adjacent lobular parenchyma, and separation of hepatic cords. |
| | Fibrous connective tissue often completely circumscribes |
| | multiple hepatic lobules, which appear irregular/shrunken. |

TABLE 3
Porcine-adapted modified METAVIR inflammation scoring scheme

| Portal inflammation/interface | Lobular necro- | |
|---|---|---|
| hepatitis | inflammatory foci | Activity score |
| None/within normal limits | None | A0 |
| | At least one per lobule | A1 |
| | Several per lobule | A2 |
| Mild to moderate | None | A1 |
| | At least one per lobule | A2 |
| Moderate with multifocal interface | None | A2 |
| hepatitis | At least one per lobule | A3 |
| Moderate to severe with marked | Any amount | A3 |
| interface hepatitis | | |

### Results

Outcome measures: The primary outcome measures of this study were effectiveness and durability of fibrosis induction. Effectiveness of fibrosis induction was measured by the occurrence of F2-F4 fibrosis at any time post-procedure, and durability of fibrosis was defined as the fraction of Cohort 2 cases exhibiting persistence of the most severe cirrhotic phenotype achieved in each individual Oncopig subject at 20-weeks post-induction. Liver cirrhosis was defined as F4 fibrosis, the same definition used clinically. Secondary outcome measures included technical success of fibrosis induction procedures, relative fibrosis compared to normal appearing liver areas and healthy control liver, rate of clinical liver decompensation, and incidence of liver lab dysfunction. Technical success was defined by proper hepatic artery catheterization with delivery of the entire prescribed dose of ethanol and ethiodized oil emulsion. Clinical liver decompensation was defined by occurrence of ascites and/or HE. Liver lab dysfunction was defined by alterations in blood chemistries compared to available laboratory reference range values, with secondary comparison to age- and sex-matched healthy control subjects.

Fibrosis induction procedures: Technical success was achieved in 9/10 (90%) cases. While all animal subjects developed reduced arterial flow during ethanol-ethiodized oil infusion as assessed under fluoroscopy, in only one case within Cohort 2, stasis in the hepatic arterial circulation required early termination of ethanol and ethiodized oil emulsion after 60% of the prescribed dose was infused (6 mL emulsion infused). A median of 10 (range 6-12) mL of absolute ethanol and ethiodized oil emulsion was administered. No procedure related complications or mortality were encountered.

Clinical and laboratory outcomes: Clinical examination revealed no evidence of liver decompensation. Neither ascites nor HE was evident in any porcine subject during the follow-up periods. Representative laboratory outcomes from select pre- and post-induction time points are displayed in Table 4. No abnormalities in platelet count, total bilirubin, alkaline phosphatase, or AST were observed among alcohol induced fibrosis and healthy control Oncopigs compared to available laboratory reference range values. Albumin levels exceeded available laboratory reference range values in Cohort 1, Cohort 2, and healthy control porcine subjects, but baseline and week 4 values in Cohorts 1 and 2 were not different than those for healthy control subjects. A statistically significant difference across week 6 albumin values (3.5 versus 3.2 versus 3.3, P=0.008) was evident. There were no available laboratory reference range values for prothrombin time, although baseline and week 4 values in Cohorts 1 and 2 were not different than those for healthy control subjects (P>0.05). A statistically significant difference across week 6 prothrombin time values (12.65 versus 12.8 versus 14.4, P=0.044) was observed. All of these porcine lab (bilirubin, albumin, and prothrombin time) and clinical (presence of ascites and hepatic encephalopathy) parameters are relevant to calculation of Child-Pugh (CP) score, a clinical staging system for liver cirrhosis. We have determined porcine equivalents to create a porcine CP system to be able to compare the induced cirrhosis status of our pigs with that observed clinically in humans.

TABLE 4
Representative laboratory outcomes

| | | |
|---|---|---|
| Cohort 1 | Cohort 2 | Control |

Platelets (10³/mL)¹

| | | | | | | |
|---|---|---|---|---|---|---|
| Baseline | 512 | (457-637) | 367 | (109-435) | 539.5 | (488-581) |
| 4-weeks | 587 | (526-750) | 340 | (235-397) | 531 | (367-549) |
| 6-weeks | 460 | (371-522) | 354 | (264-377) | 528 | (444-566) |

| | | | | |
|---|---|---|---|---|
| 8-weeks | — | 319 | (316-377) | — |
| 20-weeks | — | 301 | (298-352) | — |

Total bilirubin (mg/dL)²

| | | | | | | |
|---|---|---|---|---|---|---|
| Baseline | 0.5 | (0.3-0.9) | 0.1 | (0.1-0.3) | 0.1 | (0.1-0 2) |
| 4-weeks | 0.1 | (0.1-0.1) | 0.1 | (0.1-0.1) | 0.1 | (0.1-0.1) |
| 6-weeks | 0.1 | (0.1-0.3) | 0.1 | (0.1-0.2) | 0.1 | (0.1-0.5) |

| | | | | |
|---|---|---|---|---|
| 8-weeks | — | 0.1 | (0.1-0.3) | — |
| 20-weeks | — | 0.1 | (0.1-0.1) | — |

Alkaline phosphatase (U/L)³

| | | | | | | |
|---|---|---|---|---|---|---|
| Baseline | 217 | (200-274) | 258 | (249-293) | 166 | (127-496) |
| 4-weeks | 174.5 | (157-201) | 172 | (136-215) | 159 | (155-183) |
| 6-weeks | 135 | (120-157) | 179 | (159-232) | 146 | (106-157) |

| | | | | |
|---|---|---|---|---|
| 8-weeks | — | 193 | (150-234) | — |
| 20-weeks | — | 137 | (127-148) | — |

AST (U/L)⁴

| | | | | | | |
|---|---|---|---|---|---|---|
| Baseline | 43 | (30-52) | 46 | (29-71) | 41 | (25-50) |
| 4-weeks | 33 | (25-34) | 28 | (24-74) | 31 | (31-33) |
| 6-weeks | 35 | (19-70) | 27 | (26-182) | 40 | (26-48) |

| | | | | |
|---|---|---|---|---|
| 8-weeks | — | 20 | (16-24) | — |
| 20-weeks | — | 14 | (13-18) | — |

Albumin (g/dL)⁵

| | | | | | | |
|---|---|---|---|---|---|---|
| Baseline | 3.3 | (3.1-3.5) | 3.3 | (3.2-3.6) | 3.5 | (3.4-4.1) |
| 4-weeks | 3.6 | (3.5-3.9) | 3.6 | (3.2-3.8) | 3.7 | (3.7-3.9) |
| 6-weeks | 3.5 | (3.3-3.8) | 3.2 | (3.1-3.6) | 3.3 | (3.1-3.3) |

| | | | |
|---|---|---|---|
| 8-weeks | — | 3.7 | (3.5-3.8) |
| 20-weeks | — | 3.8 | (3.5-4.0) |

Prothrombin time (s)⁶

| | | | | | | |
|---|---|---|---|---|---|---|
| Baseline | 13.0 | (12.5-14.2) | 11.9 | (11.6-13.1) | 12.85 | (12.6-14.1) |
| 4-weeks | 13.1 | (12.2-14.4) | 12.0 | (9.5-13.1) | 14.2 | (13.2-14.3) |
| 6-weeks | 12.65 | (12.1-13.2) | 12.8 | (11.1-13.4) | 14.4 | (14.2-14.8) |

| | | | | |
|---|---|---|---|---|
| 8-weeks | — | 13.4 | (11.9-13.4) | — |
| 20-weeks | — | 11.7 | (11.4-13.5) | — |

AST = aspartate aminotransferase
¹Reference range = 300-600 103/mL (8); ²Reference range = 0-10 mg/dL (8); ³Reference range = 120-400 U/L (8); ⁴Reference range = 32-84 U/L (8); ⁵Reference range = 1.9-2.4 g/dL (8); ⁶Reference range = none available

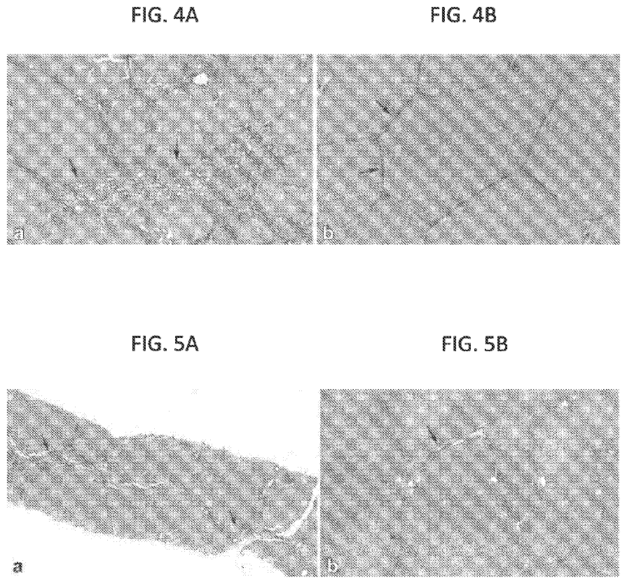

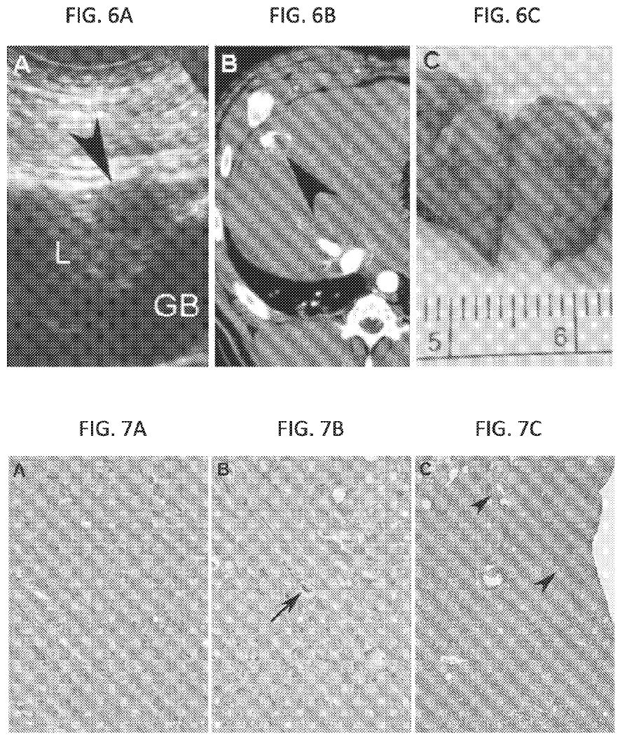

Pathological outcomes: Cohort 1. Gross pathological evaluation at necropsy revealed evidence of macronodular liver fibrosis which heterogeneously affected the liver, with areas of fibrosis and areas of normal appearing liver present in all Cohort 1 Oncopigs at 8-weeks postinduction (FIG. 3). Histopathological analysis of liver specimens collected at necropsy 8-weeks post-induction demonstrated significant fibrosis induction in all 5 Oncopigs (FIGS. 4A and 4B). Histological findings in these animals were characterized by regionally extensive foci of moderate to marked fibrous expansion of both portal tracts and pre-existing fibrous septa. Multifocally admixed with this fibrous tissue and infiltrating portal and septal tracts were moderate numbers of inflammatory cells composed primarily of lymphocytes and plasma cells, with fewer pigment-laden macrophages, and rare eosinophils. Frequently, portal inflammation and fibrosis disrupted the limiting plate and dissected into the adjacent lobular parenchyma, and often times partially or completely encircled hepatic lobules, which appeared small and irregular, and contained swollen hepatocytes undergoing lipid-type vacuolar degeneration.

Effectiveness of fibrosis induction in Cohort 1 was 100% (5/5; Table 5). At 8-weeks, the METAVIR fibrosis score for fibrotic liver from experimental animals (median F3, range F2-F4) (FIGS. 4A and 4B) was significantly higher (P=0.0013) than both normal liver from experimental animals (median F0, range F0-F1) and healthy control pigs (median F0, range F0-F1; FIGS. 4A and 4B). In addition, the inflammation score for fibrotic liver from experimental pigs (median A2, range A2-A3), was also significantly higher (P=0.0013) than both normal liver from experimental animals (median A0, range A0-A1) and healthy control pigs (median A1, range A0-A1). Median percent fibrosis (15.3%, range 5.0-22.9%) was elevated in comparison to both normal liver from experimental animals (median 6.1%, range 2.5-9.4%; P=0.080) and healthy control pigs (median 8.7%, range 5.8-12.1%. P=0.064), but did not achieve statistical significance.

TABLE 5
Cohort 1 liver histological outcomes

| | 8-week METAVIR | 8-week METAVIR |
|---|---|---|
| | fibrosis grade | inflammation activity |
| Subject 1 | 3 | A2 |
| Subject 2 | 4 | A3 |
| Subject 3 | 3 | A2 |
| Subject 4 | 4 | A3 |
| Subject 5 | 3 | A2 |
| Median | 3 | A2 |

### Samples Obtained Via Liver Tissue Harvest

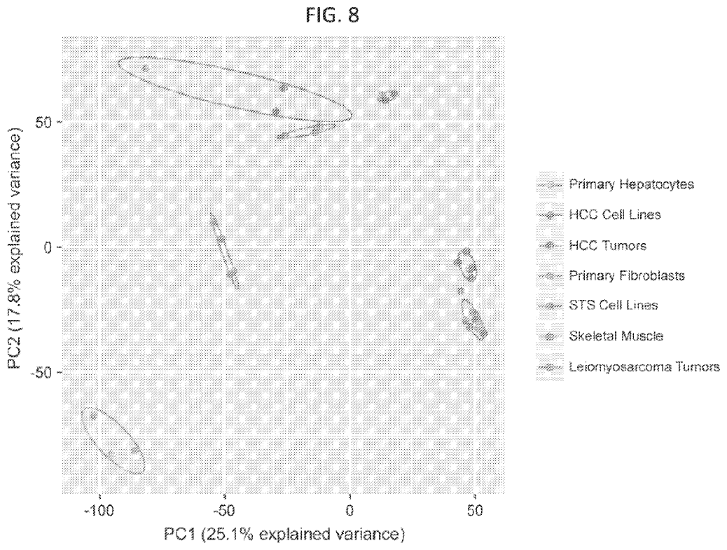

Cohort 2. Gross pathological evaluation demonstrated a normal appearance of the liver in all Cohort 2 Oncopigs at 20-weeks post-induction. Histological changes in these animals were similar to but less severe than those described for Cohort 1. Effectiveness of fibrosis induction in Cohort 2 was 100% (5/5). Histopathological examination demonstrated evidence of fibrosis as early as 2-weeks post-induction (FIGS. 5A and 5B), with a median METAVIR fibrosis score of F2 (range F1-F3), a median inflammation score of A2 (range A1-A2), and a median percent fibrosis of 8.1% (range 6.6-11.6%) at this time point.

During the follow-up period, fibrosis severity peaked at median METAVIR grade F3 (range F2-F3) and median inflammation score A2 (range A1-A2) (Table 6), but durability of fibrosis was 0% (0/5). 20-week post-induction liver histology revealed a median METAVIR fibrosis score of F2 (range F1-F2), with a reduced median inflammation score (median A1; Table 5; FIGS. 5A and 5B), but with a similar median percent fibrosis (8.7%, range 7.4-10.5%). Evaluation of serial biopsy specimens from this cohort did not demonstrate histological evidence of fibrosis progression or cirrhosis development. Some observed variation in histological results across different time points was felt to reflect sampling variation as well as fibrosis heterogeneity.

TABLE 6
Cohort 2 liver histological outcomes

| | METAVIR fibrosis grade | | METAVIR inflammation activity | |
|---|---|---|---|---|
| | Most severe^{a} | 20-week^{b} | Most severe^{a} | 20-week^{b} |
| Subject 1 | 3 (week 6) | 2 | A1 (week 2) | A1 |
| Subject 2 | 2 (week 2) | 2 | A1 (week 2) | A1 |
| Subject 3 | 3 (week 16) | 1 | A2 (week 2) | A1 |
| Subject 4 | 3 (week 2) | 1 | A2 (week 2) | A1 |
| Subject 5 | 3 (week 4) | 2 | A2 (week 2) | A1 |
| Median | 3 | 2 | A2 | A1 |

^{a}Samples obtained via percutaneous biopsy
^{b}Samples obtained via liver tissue harvest

### Example 2

### Development of Intrahepatic Hepatocellular Carcinoma in a Cirrhotic Liver Environment

### Methods

Cirrhosis induction: Cirrhosis induction was performed as described in Example 1, above. Intrahepatic tumor engraftment: Two weeks post autologous SQ tumor induction, SQ tumors were excised and dissected into roughly 1 mm segments. Utilizing a 15-gauge needle, tumor segments were inserted into the liver under ultrasound guidance. Intrahepatic tumor development was monitored every two weeks via ultrasound, and via CT 4-6 weeks post-intrahepatic tumor engraftment. See, e.g., Schachtschneider et al,Oncotarget 8(38):63620-63634, 2017. Following confirmation via CT scan, the Oncopig was sacrificed and the tumor was formalin fixed for histological evaluation.

### Results

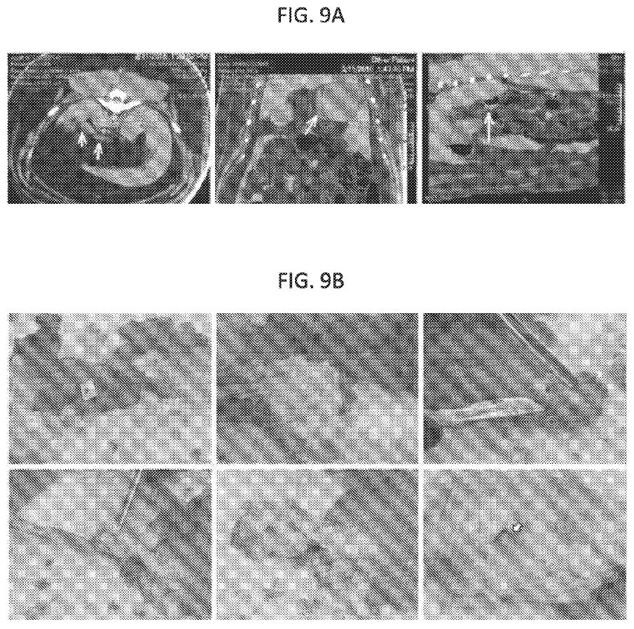

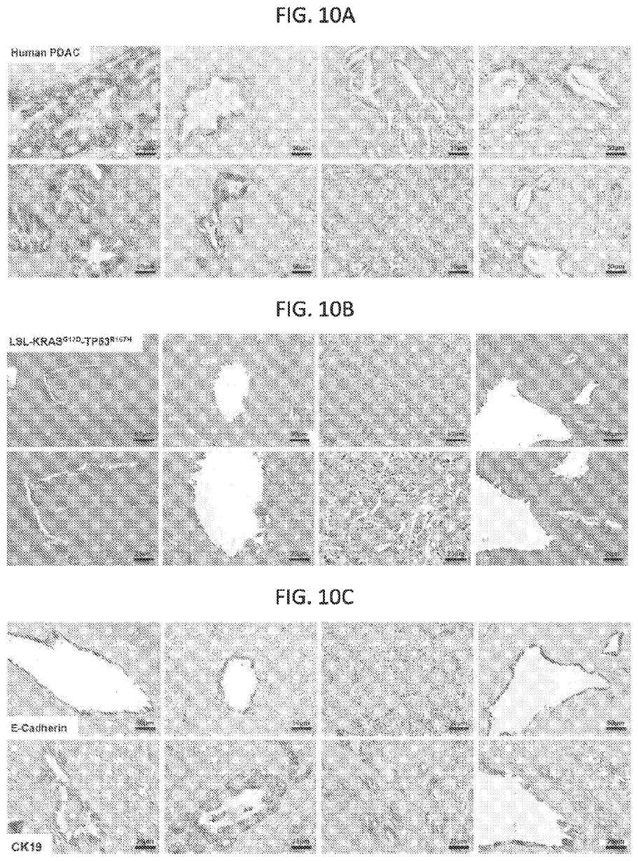

This example describes development of intrahepatic HCC tumors in cirrhotic liver microenvironments via engraftment of SQ tumor segments into the liver, which results in a 1.0 cm tumor within 30 days (FIGS. 6A-6C). The tumor mass was blindly histologically characterized by a human pathologist as HCC (with hepatocellular origin of the mass further confirmed by observation of bile in the tumor), and the surrounding liver microenvironment graded as METAVIR 2-3 liver disease (FIGS. 7A-7C). Furthermore, this highly experienced clinical pathologist specializing in the diagnosis of HCC was unable to distinguish Oncopig HCC from human HCC via histological assessment.

### Example 3

### Development of Cancers Derived from Various Organ Systems

As the OCM was designed to develop site- and cell-specific tumors in a temporal and spatial manner, additional tumors and cell lines representative of a variety of human cancers have been developed. Tumors developed to date in vivo include tumors reflective of human pancreatic ductal adenocarcinoma, soft-tissue sarcomas (STS), and HCC. The OCM cancer cell lines isolated and transformed in vitro are: fibroblasts, hepatocytes, pancreatic ductal cells, dermal epithelial cells, splenocytes, ovarian surface epithelial cells, Fallopian tube secretory epithelial cells, renal proximal tubule epithelial cells, bone marrow (no specific cell isolation), testis (no specific cell isolation), skeletal muscle (no specific cell isolation), and bladder epithelial cells. Each cell line was injected into a SCID mouse (2×10⁶ to 1×1O⁷ cells in DMEM and/or Matrigel) to determine if tumorigenic. Tumors were collected for further molecular and histologic analysis. Each of the listed cell lines has been isolated, transformed, shown to express the transgenes and form a tumor in SCID mice.

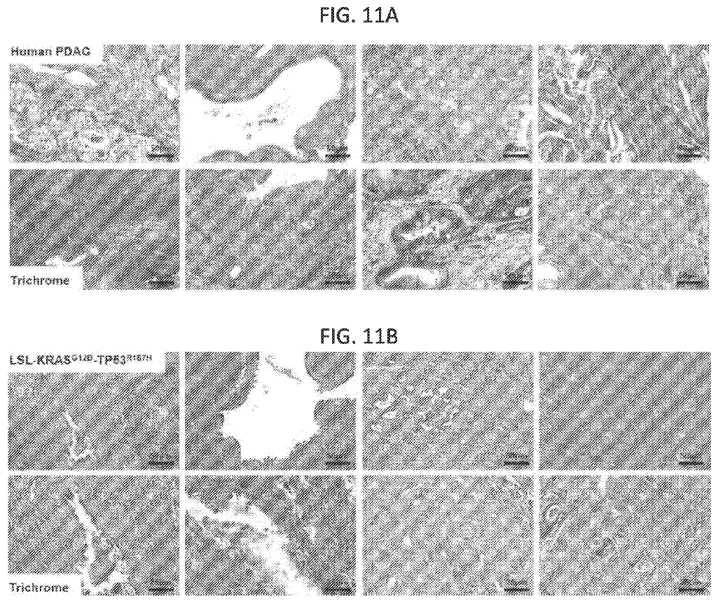

Importantly, 100% of attempted cell type isolations and transformations have been successful. In addition, although all OCM tumorigenic cells express identical genetic mutations, induced expression of these genes across cell types results in distinct expression profiles recapitulating transcriptional hallmarks of their respective human tumors (FIG. 8; Table 7).

TABLE 7
Identification of differential master regulators across OCM cancer types

| STS cells lines | Leiomysarcomas | HCC cell lines |
|---|---|---|
| FOSL1 | SPI1 | STAT1 |
| SRF | ETV4 | EP300 |
| ABCF2 | UBB | FOXA2 |
| | HMGA1 | SPI1 |
| | FOS | FOXA1 |
| | EXOSC3 | HNF4A |
| | MEF2C | HNF4G |
| | HLF | CEBPB |
| | | HNF1A |
| | | NFIC |
| | | HDAC2 |
| | | NR2F2 |
| | | NR3C1 |
| | | FOXA3 |
| | | GATA3 |
| | | E2F1 |
| | | STAT2 |

### Example 4

### Model of Pancreatic Ductal Carcinoma in the Oncopig

At least portions of this example were published in Principe et al, Scientific Reports 8: 12548, Epub Aug. 22, 2018; which is incorporated herein by reference in its entirety.

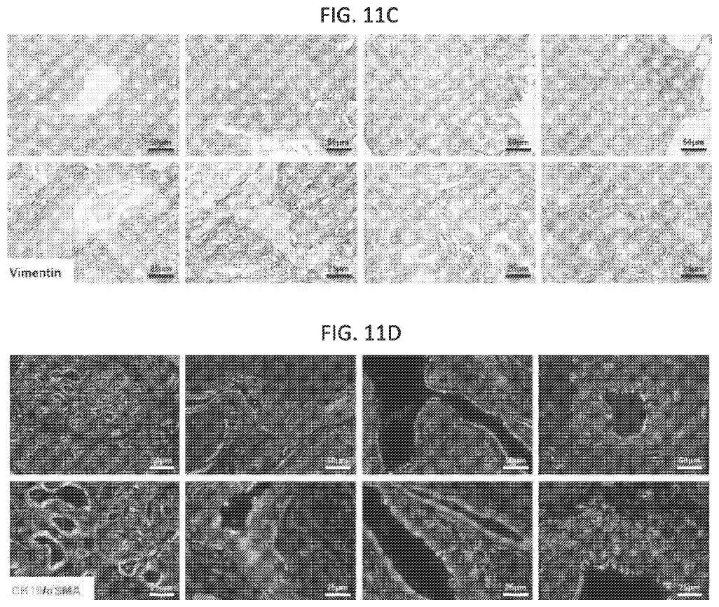

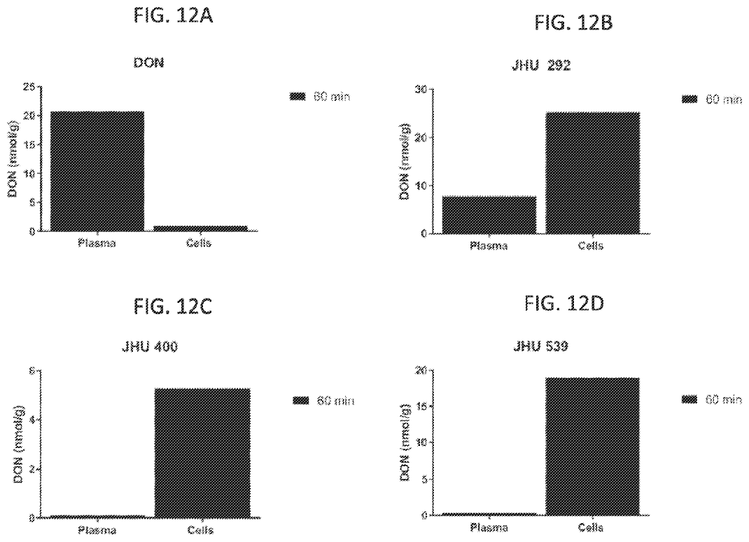

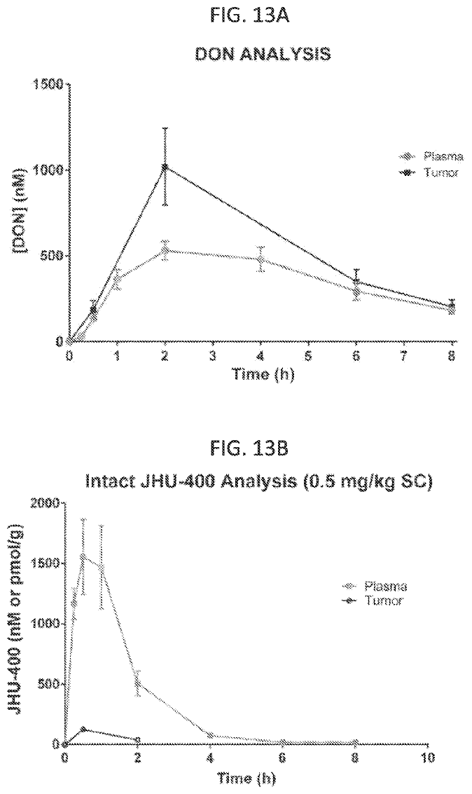

Using the OCM, in vivo pancreatic cancer that recapitulates histological hallmarks of human pancreatic cancer was induced. Injection of AdCre into the pancreatic duct resulted in several large nodules (FIGS. 9A and 9B) that recapitulated histological features of human PC, including leukocyte infiltration (FIGS. 10A-IOC) and an analogous tumor microenvironment (FIGS. 11A-1 ID).

### Example 5

### Pharmacokinetic Analysis

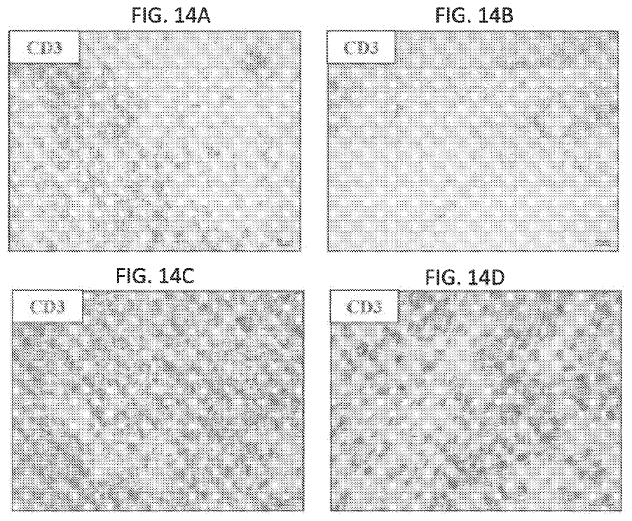

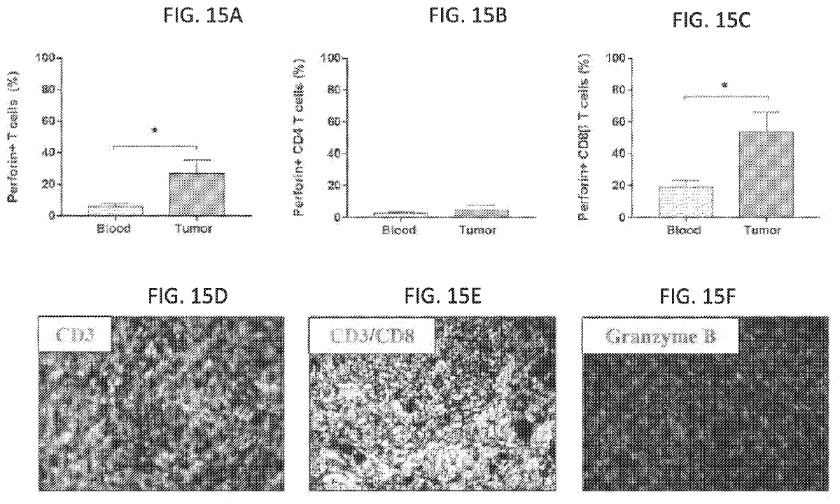

OCM soft-tissue sarcoma cell lines were used to assess tumor cell specific uptake and conversion of 6-Diazo-5 -oxo-1-norleucine (DON) prodrugs. This demonstrated the ability of the OCM to be used as a model for in vitro PK analysis (FIGS. 12A-12D). In addition, in vivo investigation of tumor specific uptake and temporal PK of systemically administered prodrugs in Oncopigs bearing SQ soft-tissue sarcomas validated the capability to employ the tumor-bearing OCM for chemotherapy drug pharmacologic studies (FIGS. 13A and 13B and Table 8). Together, these results demonstrate the value of OCM in vitro studies for prediction of in vivo results translatable to clinical practice.

TABLE 8
Selective partitioning of DON prodrugs and conversion to DON

| | | Cmax | AUC | Tumor:Plasma |
|---|---|---|---|---|
| Compound | Tissue | (pmol/ml or g) | (h*pmol/ml or g) | AUC ratio |
| DON | Plasma | 530 | 2852 | - |
| | Tumor | 1019 | 4238 | 1.5 |
| JHU-400 | Plasma | 1553 | 2931 | - |
| | Tumor | 124 | - | - |

### Example 6

### Immune Response to Tumors in the Oncopig

At least portions of this example were published in Overgaard et al, Front. Immunol. 9:1301, Epub Jun. 7, 2018; which is incorporated herein by reference in its entirety.

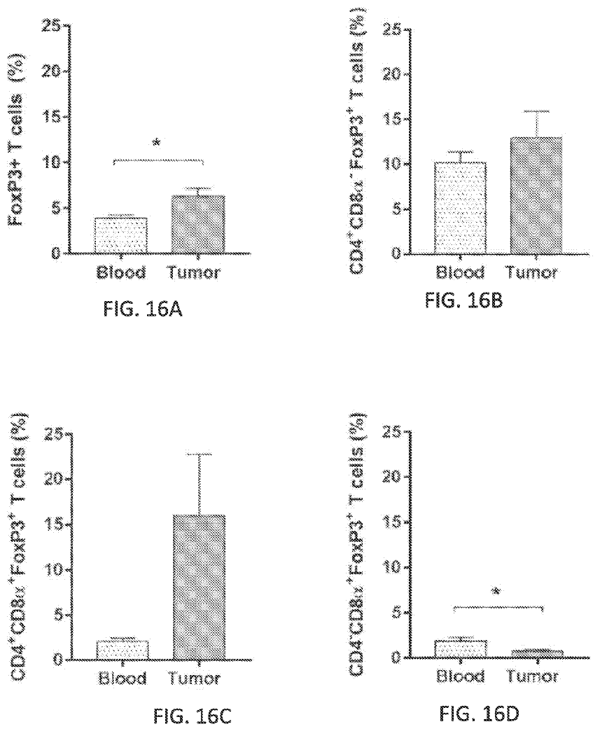

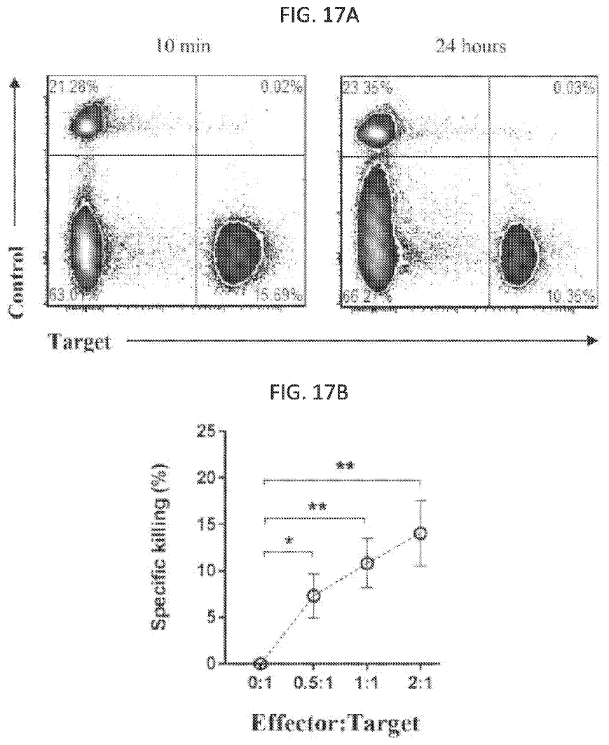

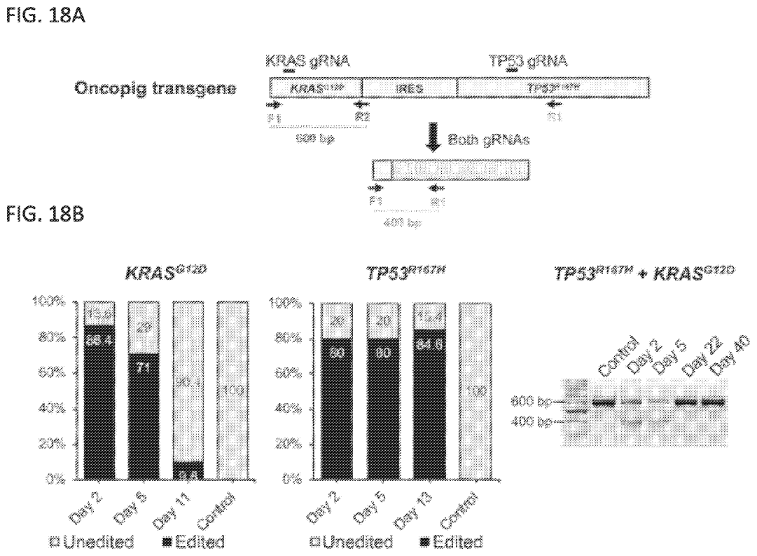

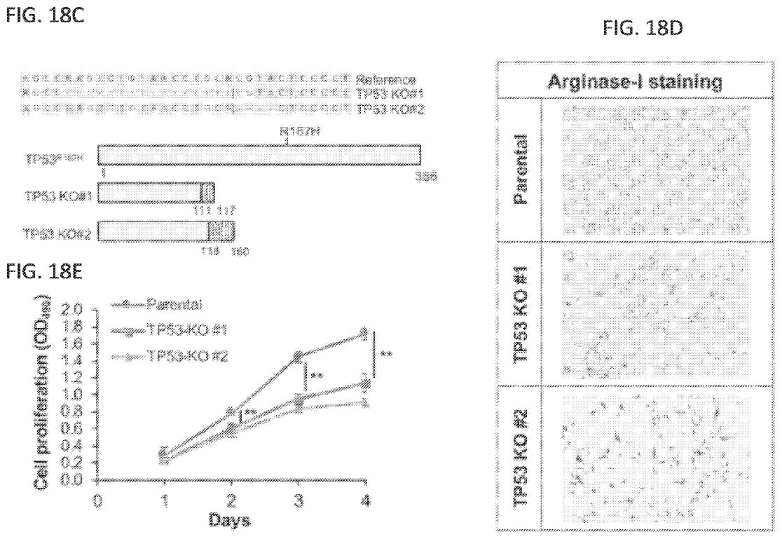

Oncopigs were subcutaneously injected with AdCre and sarcomas formed 7-21 days post-injection. Immune profding was performed, confirming the presence of pronounced intratumoral T-cell infiltration (FIGS. 14A-14D), including cytotoxic (FIGS. 15A-15F) and regulatory (FIGS. 16A-16D) T-cell infiltration, expression of immune checkpoints (RNA-seq; Table 9), and cytotoxic responses against tumor cells (FIGS. 17A and 17B). Together, these results support the use of the OCM as a valuable model for preclinical testing of immunotherapies aimed at reactivating tumor-directed cytotoxicity in vivo.

TABLE 9
Elevated IOD1, CTFA4, and PDF1 expression in Oncopig sarcoma

| | Skeletal | | Log2 | | | |
|---|---|---|---|---|---|---|
| | Muscle | Sarcoma | fold | | | |
| Gene | (FPKM) | (FPKM) | change | p-value | q-value | Significant? |
| IDO1 | 0.49 | 3.80 | 2.96 | 5.00E−05 | 0.00023 | Yes |
| CTLA4 | 0.13 | 1.02 | 2.96 | 5.00E−05 | 0.00023 | Yes |
| PDL1 | 0.34 | 1.09 | 1.66 | 0.00075 | 0.00276 | Yes |

### Example 7

### Development of Tumors from Genetically Defined Genome Edited HCC Cell Lines

### Methods

CRISPR-Cas9 gene editing: Since the Oncopig transgenes are inserted as cDNA, gRNAs targeting an exon -exon junction in each transgene TP53^{R167H} or KRAS^{312D}) as well as the ARID 1A and AXIN1 genes were designed using the CRISPOR web tool. Each gRNA was synthesized by incubating equimolar ratios of Alt-R™ crRNA and tracrRNA (#1072532; IDT Corporation, Chicago, IF) at 95° C. for 5 minutes and cooling to room temperature. Each gRNA was combined with purified S. pyogenes Cas9 nuclease (#1081058; IDT Corporation) to form a ribonucleoprotein (RNP) complex. Oncopig HCC cells were reverse transfected with 25 nM RNP complexes using the Fipofectamine CRISPRMAX kit (#CMAX00003; Invitrogen) following the manufacturer's instructions.

Confirmation of gene editing: Genomic DNA was extracted from genome edited Oncopig HCC cells using QuickExtract DNA Extraction Solution (#QE09050; Fucigen, Middleton Wis.) following the manufacturer's instructions. The genomic locus that flanks the Cas9 target site was amplified by PCR. A second PCR was performed to attach Fluidigm adaptor and barcode sequences. Targeted sequencing was performed using a MiSeq (Illumina, San Diego, Calif.) following the manufacturer's instructions. Sequencing reads were analyzed using CRISPResso2 with default parameters.

### Results

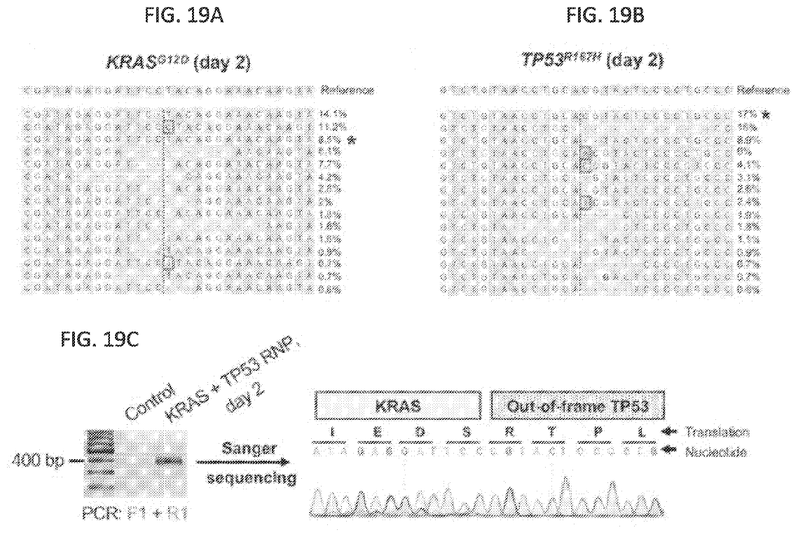

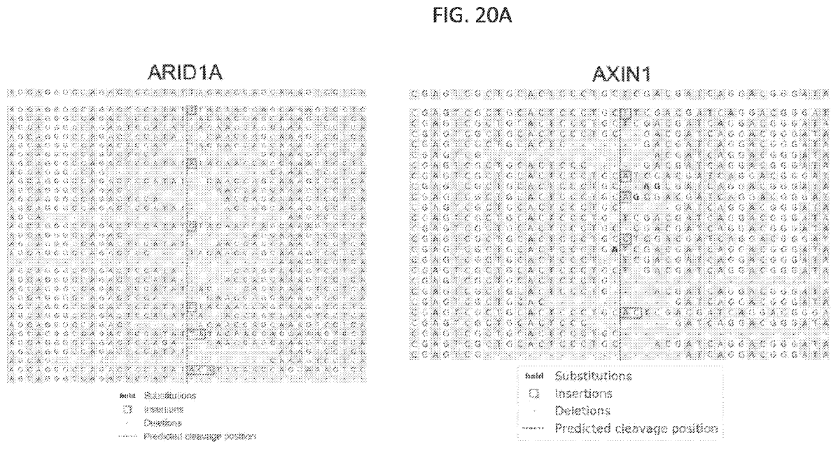

Oncopig HCC cell lines are genetically manipulatable: Advances in animal modeling and gene editing provide an opportunity to develop genetically tailored tumors that reflect what is observed clinically. This enables investigation of the contribution of clinically relevant driver mutations on tumor progression and treatment susceptibility, as well as preclinical testing of novel precision medicine approaches. As a first step towards generation of genetically tailored Oncopig HCC tumors, the ability to knockout (KO) the Oncopig TP53^{KI27″} and KRAS′¹²¹¹ driver mutations was tested using CRISPR-Cas9 (FIG. 18A). Oncopig HCC cells were successfully edited at a rate of over 80% (FIG. 18B), with insertions or deletions (INDELs) occurring around the predicted cleavage sites (FIGS. 19A and 19B). In addition, simultaneous targeting of TP53 [symbol] and KRAS^{G12D} resulted in deletion of the region between the two gRNAs (FIGS. 18A and 18B), which was confirmed via Sanger sequencing (FIG. 19C). While the percentage of [symbol] edited cells was maintained in culture for up to two weeks, the proportion of cells harboring KRAS^{G12D} edits decreased over time (FIG. 18B), suggesting KRAS^{G12D} is required for Oncopig HCC cell survival. Isolation and screening of 5 single cell clones from the TP53 [symbol] edited cell pool resulted in development of two TP53 [symbol] KO HCC cell lines harboring frameshift mutations (17 and 4 bp deletions, respectively) leading to protein truncation (FIG. 18C). The parental and TP53 [symbol] KO cell lines stained positive for arginase-1 (FIG. 18D), confirming their identity as HCC cells. As expected, TP53 [symbol] KO resulted in reduced cell proliferation compared to the parental line (FIG. 18E), demonstrating the ability to introduce genetic alterations with significant effects on malignant potential.

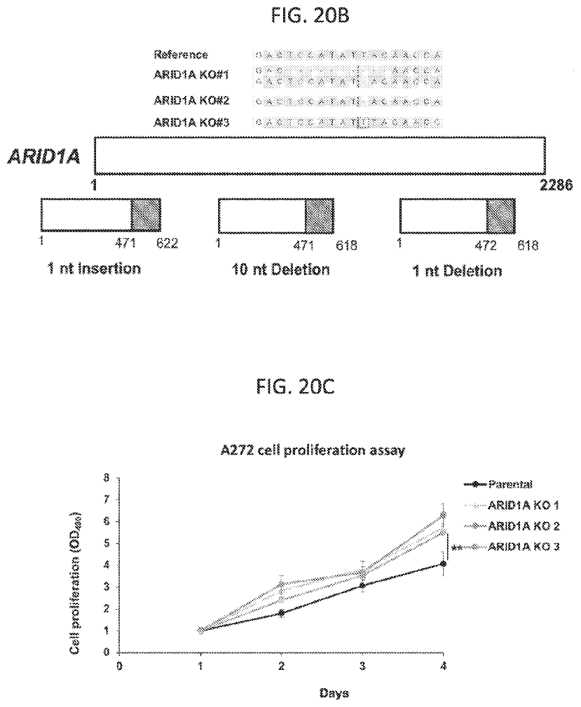

Following confirmation that driver mutations can be successfully removed from Oncopig HCC cells, the ability to introduce additional driver mutations was tested through the knockout (KO) of ARID1A and AXINI, which represent tumor suppressor genes commonly mutated in 10-15% of HCC tumors. This resulted in successful ARID1A and AXINI editing in the Oncopig HCC cell lines as determined by targeted Illumina sequencing (FIG. 20A). Single cell clones were then isolated and screened for ARID1A mutations, resulting in successful isolation of 35 single cell clones, 18 of which harbored ARID 1A KO mutations. The functional effect of ARID 1A KO was evaluated in 3 ARID 1A KO single cell clones (FIG. 20B). Consistent with its reported tumor suppressor role, ARID 1A KO increased Oncopig HCC cell proliferation (FIG. 20C). Following in vitro characterization, a pool of ARID1A edited Oncopig HCC cells were autologously injected subcutaneously into an Oncopig. A tumor mass measuring 2 cm developed within two weeks. The subcutaneous mass was then collected for histological and genomic analyses. Confirmation that the subcutaneous mass was an HCC tumor harboring ARID1A mutations was confirmed by histological analysis and targeted Illumina sequencing.

Together, these results provide proof of concept data for combining the Oncopig orthotopic HCC model with in vitro gene editing to develop genetically tailored HCC tumors for investigating the contribution of driver mutations on clinically relevant cancer phenotypes and testing of novel precision medicine approaches.

### Example 8

### Chemotherapeutic Screening

Cells (1×1O⁴ cells/well) were cultured in 200 [symbol] DMEM+10% FBS overnight in 96 well plates. The following day, culture media was replaced with fresh media supplemented with one of 10 varying concentrations of each chemotherapeutic compound. Sorafenib, doxorubicin, mitomycin C, and 5-Fluorouracil (5-FU) were prepared from a stock solution in a 10-point serial dilution curve, in DMSO, and added at 1:100 to culture media (final concentration of DMSO was 1% for all conditions). As platinum complexes are inactivated by DMSO, cisplatin stock solution was 3.33 mM in water and the stock was used to prepare all dilutions. Sorafenib and mitomycin C were tested from 0.5-100 μM, doxorubicin from 0.1-20 μM, cisplatin from 1-200 μM, and 5-FU from 1-500 μM in order to assay concentrations that are relevant for clinically used dosages and that allow for calculation of the half maximal inhibitory concentration (IC50) for each compound. 96 well plates with each cell line and chemotherapeutic concentration in triplicate were prepared for time points 0, 24, 48, and 72 hours to observe a dose response overtime. For the 3-(4,5-dimethylthiazol-2-yl)-2,5-diphenyltetrazolium bromide (MTT) assay, 100 [symbol] of fresh media was replaced at each time point, 10 [symbol] of 5 mg/ml MTT added to each well and incubated for 4 hours at 37° C. 100 [symbol] for an SDS/HCl solution (1 g SDS/10 ml 0.1 HCl) was then added to solubilize the resulting formazan dye and the plates incubated an additional 4 h at 37° C. Absorbance at 570 nm was read using a BioTek 800 TS Absorbance Reader (BioTek). Results are expressed in percentage, relative to the absorbance values for each cell line at time 0.

OCM and human HCC cell lines display similar sensitivities to four FDA approved cytostatic (sorafenib) and cytotoxic (doxorubicin, cisplatin, mitomycin C) chemotherapeutic agents used to treat HCC clinically, either systemically or via transarterial chemoembolization (TACE), and one shown to be ineffective (5-FU) in clinical settings. OCM and human (HepG2) HCC cell lines display analogous IC50 values following exposure to comparable, clinically relevant concentrations for 72 hours (Table 10). Although chemotherapeutic responses were comparable between OCM and human lines, murine HCC (Hepal-6) lines displayed significantly higher (10 fold) susceptibility to 5-FU, which has proven to be ineffective in clinical practice. These results support the concept that OCM HCC can be used to more effectively screen and test promising drugs than current HCC models, with results that are translatable to clinical practice.

TABLE 10
OCM, human and murine IC50 (μM)

| Cell line | Doxorubicin | Mitomycin C | Cisplatin | Sorafenib | 5-FU |
|---|---|---|---|---|---|
| OCM | 0.08 | 0.48 | 5.38 | 10.36 | 14.51 |
| MCC | | | | | |
| HepG2 | 0.23 | 0.57 | 5.6 | 8.86 | 18.28 |
| Hepal-6 | 0.53 | 0.61 | 2.16 | 4.98 | 1.82 |

### Example 9

### Alcohol Feeding Improves Clinical Relevance of Porcine Liver cirrhosis Model

### Methods

Fibrosis induction: Fibrosis inductions were performed for 2 Oncopigs. All fibrosis induction procedures were performed by a board-certified Interventional Radiology (IR) physicians according to a modification of the methodology described by Avritscher et al. (J Vase. Interv. Radiol. 22: 1329-1334, 2011). At 12-weeks of age, Oncopigs underwent anesthetic induction, followed by intubation and maintenance with 1-3% isoflurane. Angiography was performed using a C-arm (OEC Medical Systems series 9600; GE Healthcare, United Kingdom). With the animal supine, the groin was sterilely prepared. Ultrasound-guided vascular access was gained via the common femoral artery with placement of a 5 French sheath (Pinnacle; Terumo Medical Corporation, Somerset N.J.). Using standard catheter and wire techniques, celiac arteriography was performed using a 5 French catheter (Sos Omni Selective; AngioDynamics, Latham N.Y.). A coaxial 3 French microcatheter (Renegade Hi-flo; Boston Scientific, Marlborough Mass.) was advanced into the common hepatic artery using a micro -guidewire. Arteriography was performed with iohexol (Omnipaque-300; Amersham Health, Princeton N.J.). The microcatheter was then advanced into the proper hepatic artery, and 0.75 mL/kg of a 1:3 v/v emulsified mixture of absolute ethanol and ethiodized oil (Lipiodol; Guerbet, Villepinte France, used to deliver ethanol into the liver and transiently embolize the liver microcirculation) was slowly infused by hand injection over 30 minutes. Dosing of the administered ethanol and ethiodized oil emulsion was derived by dividing the 28 mL maximally tolerated dose by mean pig weight reported in the study of Avritscher et al. (J. Vase. Interv. Radiol. 22: 1329-1334, 2011). Upon completion of the infusion, all devices were removed, and hemostasis was achieved via manual compression.

Alcohol feeding: Following cirrhosis induction, both Oncopigs were provided 10% ethanol mixed with KoolAid and Swine BlueLite daily at a rate at which 40% of their daily caloric intake was provided by the ethanol. The ethanol mixture was provided every morning in a bottle, and pigs were allowed to drink throughout the day. Both pigs began drinking 100% of the provided ethanol within the first week, and continued to drink for the duration of the study (2 months). Liver biopsies were collected at 1 and 2 months post cirrhosis induction, formalin fixed, and trichrome stained. Biopsies from alcohol fed pigs were compared to samples from a previous study where pigs underwent cirrhosis induction without alcohol feeding (Example 1).

### Results

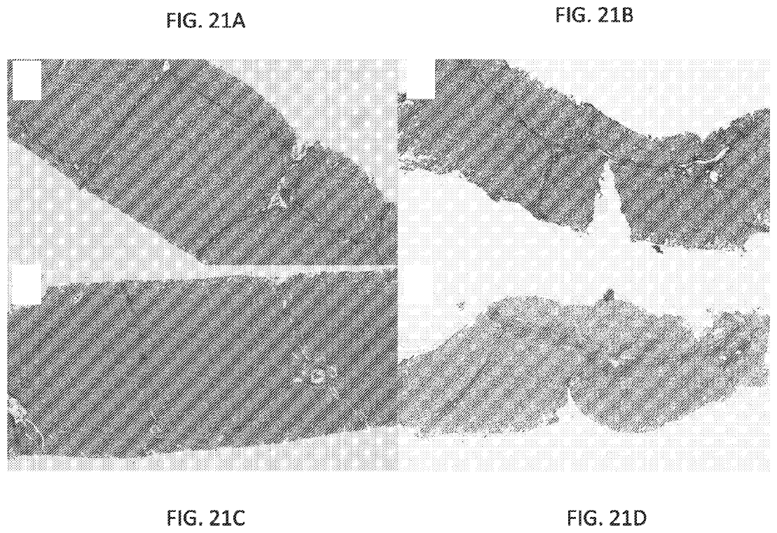

Previous studies have demonstrated that while transarterial administration of ethanol and ethiodized oil into the hepatic circulation results in METAVIR stage F2-F3 fibrosis with moderate inflammation within 8 weeks. However, without continued alcohol exposure, Oncopig livers quickly recover and display histological phenotypes similar to a normal liver by 20 weeks post induction. In an attempt to increase the disease severity and prolong the disease phenotype, the cirrhosis induction protocol was combined with daily feeding of 10% ethanol at a rate of 40% of daily caloric intake. Histological results from biopsies collected 4- and 8-weeks post cirrhosis induction demonstrate increased fibrosis and hepatocyte damage in alcohol fed pigs compared to non-alcohol fed pigs (FIGS. 21A-21D). The alcohol feeding also provides chronic alcohol exposure, which more closely mimics the clinical presentation of patients with alcohol-induced liver cirrhosis.

### Example 10

### Personalized Tumor Modeling

Utilizing the Oncopig approach, personalized tumor models are developed to facilitate performance of a trial on a patient's tumor. Through a combination of tumor and comorbidity induction as described above, animal cohorts representative of individual patients are developed to screen for optimal treatment strategies for that individual. This personalized medicine optionally utilizes gene-editing technologies to develop tumors with driver mutations representative of the individual's tumor genotype.

Exemplary protocols for in vitro and in vivo models are provided. One of ordinary skill in the art will recognize that modifications to these methods can also be made to produce personalized tumor models. The process of developing cohorts of personalized tumor models can be undertaken in as little as 6 months, which is substantially less time than average trial activation and first subject enrollment for human clinical trials. This model allows for rapid testing of personalized treatments to be translated to clinical practice.

An in vitro protocol includes collecting a tumor sample from a patient, extracting DNA from the tumor sample, and identifying mutations of interest, for example by sequencing. The mutation(s) are introduced into pig cells in vitro using gene editing approaches (for example, using CRISPR techniques). The ability to induce CRISPR-Cas9 gene edits in Oncopig HCC cells has been demonstrated (see Example 7, above). The pig cells can be already cancerous (for example Oncopig cells exposed to Cre recombinase to induce transgene expression) or noncancerous (primary) pig cells. Introduction of these mutations into primary cells will render the cells tumorigenic. The modified cells are injected into a pig (e.g., using the cancer cell injection protocols described herein, such as into the organ of interest, s.c. injection followed by implantation to organ of interest, or systemic delivery described for metastatic disease). The tumor is then grown and interventions are tested (for example, as described in Section VI).

An in vivo protocol includes collecting a tumor sample from a patient, extracting DNA from the tumor sample, and identifying mutations of interest, for example by sequencing. The mutation(s) are introduced into pig cells in vitro using gene editing approaches (for example, using CRISPR techniques), either in an existing tumor or by introducing the mutation(s) in normal tissue. Introduction of mutations into normal tissue will render the edited cells tumorigenic, resulting in tumor formation. The tumor is then grown and interventions are tested (for example, as described in Section VI).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1-34. (canceled)

35. A method of enhancing duration and severity of liver cirrhosis in a pig, in which liver fibrosis has been induced by infusing ethanol into the common hepatic artery of the pig, comprising:
feeding the pig dietary ethanol in an amount and for a duration sufficient to produce more sustained and more severe liver fibrosis compared to ethanol infusion alone.

36. The method of claim 35, wherein the pig is a transgenic pig.

37. The method of claim 36, wherein the transgenic pig is transgenic for Cre recombinase-inducible KRAS^{G12D} and TP53^{R167H}.

38. The method of claim 35, further comprising producing cancer cells from the pig.

39. The method of claim 38, wherein the cancer cells are hepatocellular carcinoma (HCC) cells.

40. The method of claim 37, further comprising producing cancer cells from the pig.

41. The method of claim 40, wherein the cancer cells are hepatocellular carcinoma (HCC) cells.

42. The method of claim 35, wherein the dietary ethanol comprises about 40% of total caloric intake.

43. The method of claim 42, wherein the duration of feeding the pig the dietary ethanol is about four weeks.

44. The method of claim 42, wherein the duration of feeding the pig the dietary ethanol is about four to eight weeks.

45. The method of claim 40, further comprising modeling one or more therapies for cancer treatment.

46. The method of claim 45, wherein modeling the one or more therapies comprises:
screening a potential therapeutic compound by contacting HCC cells from said pig with said compound to determine if cell growth and survival is affected by said compound.

47. The method of claim 45, wherein the modeling the one or more therapies comprises:
identifying a personalized cancer treatment for a patient in which the genome of tumor cells from said patient has been sequenced to identify one or more mutations that are introduced into HCC cells from said pig in order to test potential anti-oncolytic therapies thereon.
